(19) United States
(12) Patent Application Publication
(10) Pub. No.: US 2024/0216437 A1
(43) Pub. Date: Jul. 4, 2024

(54) METHODS AND COMPOSITIONS FOR TREATING OCULAR GRAFT VERSUS HOST DISEASE AND OTHER OCULAR CONDITIONS

(71) Applicant: MAM Holdings of West Florida, L.L.C., Palm Harbor, FL (US)

(72) Inventor: Carl Randall Harrell, Tarpon Springs, FL (US)

(21) Appl. No.: 18/543,748

(22) Filed: Dec. 18, 2023

Related U.S. Application Data
(60) Provisional application No. 63/435,877, filed on Dec. 29, 2022.

(51) Int. Cl. A61K 35/28; A61K 45/06; A61P 27/02

(52) U.S. Cl. CPC A61K 35/28; A61K 45/06; A61P 27/02

(57) ABSTRACT

Disclosed herein are compositions, formulations, and/or methods of using d-MAPPS™ regenerative biologics platform technology (“d-MAPPS”). Specifically, d-MAPPS compositions, which may include d-MAPPS solutions (e.g., in liquid form and/or administered as eye drops), can be used for topical application to the eye, which is useful for the treatment of ocular conditions (e.g., dry eyes, Sjogren's syndrome, cataracts, burns, and injuries to the eye tissues). The d-MAPPS compositions can contain mesenchymal stem cells (MSC), MSC-derived exosomes (MSC-Exos), and/or one or more MSC-sourced growth factors and/or immunoregulatory proteins. In at least other embodiments, the d-MAPPS compositions can include a sterile de-cellularized human amniotic fluid (D-HAF). The d-MAPPS compositions are amenable for long-term storage without the loss of biological potency. Methods for treating, or preventing various ocular diseases, injuries and disorders using the formulation, optionally in combination with one or more therapeutic, prophylactic or diagnostic agents are described.

### CROSS-REFERENCE TO RELATED APPLICATION

This application claims priority to U.S. Provisional Application No. 63/435,877, filed Dec. 29, 2022, which is hereby incorporated by reference in its entirety.

### STATEMENT REGARDING FEDERALLY SPONSORED RESEARCH

This invention was made without government support or grants awarded by the National Institutes of Health. The government has no rights in the invention.

### FIELD OF THE DISCLOSURE

The present disclosure relates to methods and composition for treating diseases of the eye and other disorders. In particular, embodiments of the disclosure relate to methods and compositions for the prevention, management, and treatment of various ophthalmic diseases, ocular injuries, and other disorders. In certain embodiments, therapeutic compositions may include, for example, mesenchymal stem cells (MSC), MSC-sourced growth factors, and various immunoregulatory proteins.

### BACKGROUND

Diminished vision has a direct causal impact on a person's ability to perform basic functions such as walking, reading, driving and other common activities. Ocular health contributes significantly to a person's quality of life and their ability to perform common daily activities. Disorders and diseases of the eyes can be severely incapacitating, and may occur in patients in a wide variety of forms. Millions of people suffer from some form of visual impairment.

As an example, dry eye, a common ocular disorder sometimes related to autoimmune disorders, the formation of cataracts and aging in general, afflicts tens of millions of people globally. The lives of people with dry eye are negatively impacted due to consistent pain, redness and/or dryness of the eyes. Typically, artificial tears and over-the counter eye drops are used by patients to sooth eye irritation and to lubricate the eyes. The majority of these treatment options provide minimal relief for limited duration and require several daily reapplications. These treatment options provide varying degrees of relief with limited capacity to modify the underlying disease state.

Human amniotic membrane (HAM) has been used efficaciously to treat specific eye surface injuries and maladies. However, the use of HAM often involves the skills of a physician and additional expense to patients.

Additionally, these procedures usually impose severe vision impairment during treatment as the amniotic membrane is non-transparent. Ultimately, the benefits of the procedure last only as long as the membrane is in place, so the procedure is not particularly useful for chronic conditions such as dry eye.

Mesenchymal stem cells (“MSC” or “MSCs”) are self-renewable, multipotent stem cells that regulate innate and/or adaptive immune responses in various human tissues. For instance, MSCs play a role in responding to tissue injury and reducing inflammation. Moreover, due to their immunosuppressive properties, MSCs have therapeutic potential in alleviating various diseases (e.g., ophthalmic diseases, ocular diseases, autoimmune diseases, and specific cancers). MSCs may originate from different sources (e.g., bone marrow, amniotic fluid, placental tissue, etc.) and contain a variety of biological compounds (e.g., carbohydrates, proteins and peptides, lipids, lactate, pyruvate, electrolytes, enzymes, hormones, and various growth factors).

Given the foregoing, there is a significant need for compositions and formulations that can be used for the management of various eye diseases, injuries and disorders, and that are affordable, readily accessible, and easy to use for both clinician and patient. In particular, there is a need for compositions and/or formulations that provide for the clinical use of MSCs and/or MSC-derived products (e.g., exosomes, growth factors, immunoregulatory proteins, etc.) in preventing, managing, and/or treating various eye diseases.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In certain embodiments, the disclosed embodiments may include one or more of the features described herein.

Embodiments of the present disclosure are directed towards compositions, formulations, and methods for using one or more types of mesenchymal stem cells (“MSC” or “MSCs”), and/or products derived therefrom (e.g., exosomes or growth factors derived therefrom), for preventing, managing, and/or treating various ophthalmic and ocular diseases. Specifically described herein is an MSC-derived biological product d-MAPPS™ regenerative biologics platform technology (“d-MAPPS”), which contains large numbers of MSC-sourced growth factors, anti-inflammatory cytokines and chemokines. Notably, the acronym d-MAPPS in d-MAPPS™ regenerative biologics platform technology stands for “derived Multiple Allogeneic Proteins Paracrine Signaling”. In vitro, d-MAPPS™ regenerative biologics platform technology efficiently inhibits proliferation of activated human peripheral blood mononuclear cells (“pbMNCs”). d-MAPPS™ regenerative biologics platform technology further suppresses production of inflammatory cytokines and promotes secretion of immunosuppressive factors in pbMNCs. In addition, d-MAPPS™ regenerative biologics platform technology favors development of tolerogenic and regulatory phenotype in activated monocytes and lymphocytes, indicating its potential for therapeutic use in various diseases, including one or more of the eye diseases described herein (e.g., dry eye, graft-versus-host disease, ocular graft-versus-host disease), as well as other diseases (e.g., various cancers).

In at least one embodiment, d-MAPPS™ regenerative biologics platform technology includes immunostimulatory molecules (e.g., IL-27 and CXCL16) that enhance T-cell driven immune responses. In some embodiments, a method for prevention and treatment of a disease (e.g., one or more eye diseases) is disclosed, including altering the response of endogenous immune cells in the subject provided, comprising administering to the subject an effective amount of d-MAPPS™ regenerative biologics platform technology, thereby altering the response of endogenous immune cells (e.g., dendritic cells, macrophages, natural killer cells, T cells, and the like) in the subject. In embodiments, administration of effective amount of d-MAPPS™ regenerative biologics platform technology improves one or more symptoms of one or more eye diseases in the subject. In some embodiments, d-MAPPS™ regenerative biologics platform technology may be administered in combination with one or more agents selected from the group consisting of d-MAPPS-associated MSCs, placenta tissue-derived MSCs, antimicrobial agents, analgesic agents, local anesthetic agents, anti-inflammatory agents, anti-oxidant agents, immunosuppressant agents, anti-allergenic agents, enzyme cofactors, essential nutrients, growth factors, and combinations thereof.

In some embodiments, a pharmaceutical composition comprising d-MAPPS™ regenerative biologics platform technology (also referred to herein as “d-MAPPS pharmaceutical composition”) is disclosed. Such d-MAPPS pharmaceutical compositions may be formulated in various formulations, including, for example, as one or more liquid solutions (also referred to herein as “d-MAPPS solution” or “d-MAPPS solutions”). At least one such d-MAPPS solution is suitable for administration to the eyes of a subject as eye drops. Other formulations (e.g., gels, solids) are possible. The d-MAPPS pharmaceutical compositions may comprise, for instance, one or more types of MSCs, one or more types of exosomes derived from one or more types of MSCs (“MSC-Exos”) (e.g., exosomes generated ex vivo from mesenchymal stem cells, wherein the mesenchymal stem are placental tissue-derived mesenchymal stem cells), one or more MSC-sourced growth factors and/or immunoregulatory proteins, and/or sterile de-cellularized human amniotic fluid (D-HAF). Such exosomes may be used as a delivery vehicle for one or more MSC-sourced biological molecules (e.g., anti-tumorigenic miRNAs, messenger RNAs (mRNAs), enzymes, cytokines, chemokines, growth factors, immunomodulatory factors, small-molecule drugs, proteins, and combinations thereof. The d-MAPPS pharmaceutical compositions may further comprise one or more pharmaceutically acceptable excipients. The d-MAPPS pharmaceutical compositions may also comprise one or more agents selected from the group consisting of adjuvants, antioxidants, anti-inflammatory agents, growth factors, neuroprotective agents, antimicrobial agents, local anesthetics, and combinations thereof.

In at least one example, the d-MAPPS pharmaceutical composition includes a formation for topical application to the eye for the treatment of ocular diseases and injuries including dry eyes, Sjogren's syndrome, cataracts, burns, and injuries to the eye tissues. The aforementioned composition may, in some instances, contain a human amniotic fluid formulation. Such human amniotic fluid formulation may be a specifically formulated, sterile filtered de-cellularized human amniotic fluid. The d-MAPPS pharmaceutical composition may be applied directly to the eye(s), preferably as a liquid ocular solution (e.g., one or more d-MAPPS solutions), much like a common liquid eye drops, lubricant, or gel. The d-MAPPS pharmaceutical composition can alleviate or prevent at least one symptom of a number of ocular injuries and diseases, including chronic dry eye disease, Sjogren's syndrome, and burns or injuries, corneal neovascular disorders, corneal opacities (including corneal haze), and prolonged redness and inflammation of the eye(s).

Specifically, a d-MAPPS pharmaceutical composition may include a sterile de-cellularized human amniotic fluid (D-HAF), preferably diluted with a pharmaceutically accepted carrier, and typically administered using a standard eye dropper apparatus. D-HAF contains over 300 human growth factors. D-HAF is devoid of amniotic stem cells and elements of micronized membrane or chorion particles. The dilution ratio of the D-HAF is dependent on the severity of the disorder or injury. For example, early to moderate dry eye or chronic redness, surface inflammation and, intraocular inflammation may be best treated with a low concentration, whereas Sjogren's syndrome, severe Dry Eye, a corneal neovascular disorder, or corneal opacity will typically utilize a higher concentration of D-HAF. Daily applications of a d-MAPPS pharmaceutical composition (e.g., a composition containing D-HAF) can deliver a sustainable level of beneficial growth factors.

D-HAF is prepared from human amniotic fluid (“AF”) from which the amniotic stem cells and particulate matter have been removed. In at least one embodiment, the process includes separating the cells from the AF using centrifugation and utilizing a series of filtration devices to remove all remaining cells and bioburden. Each lot is tested for bioburden and is certified sterile to contain <1 harmful organisms. The purified fluid is sterilized without the use of harsh terminal irradiation, e-beam, or Ethylene Oxide (EO).

Methods for treating or preventing an ocular disease, disorder, or injury of the eye using one or more of the described d-MAPPS pharmaceutical compositions (e.g., one or more d-MAPPS solutions) are described. In some embodiments, the aforementioned compositions are administered with a pharmaceutically acceptable carrier. In some embodiments, such compositions are administered as a solution, suspension, ointment, or gel, with or without an implant. In some embodiments, the disorders associated with the eye that are suitable for treatment include dry eye disease, ocular burns, tears or injury to the eye or associated structures, corneal neovascular disorders, corneal opacities (including corneal haze), ocular blast injuries, eye infections, eye surgeries, drug-induced eye conditions, and prolonged redness and inflammation of the eye. In some embodiments, the disorders to be treated include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, anterior uveitis, posterior uveitis (including toxoplasmosis), pan-uveitis, an inflammatory disease of the vitreous or retina, endophthalmitis prophylaxis and treatment, macular edema, macular degeneration, age related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma, and combinations thereof. Other disorders include injury, burns, or abrasion of the cornea, cataracts and age related degeneration of the eye or vision associated therewith.

Methods for treating, or preventing a disease, disorder, or injury of the eye using one or more d-MAPPS pharmaceutical compositions (e.g., one or more d-MAPPS solutions) in combination with one or more therapeutic, prophylactic or diagnostic agents are also described. In some embodiments, one or more d-MAPPS pharmaceutical compositions is administered prior to, in conjunction with, subsequent to, or alternation with treatment with one or more therapeutic, prophylactic or diagnostic agents. In some embodiments, the one or more therapeutic, prophylactic or diagnostic agents are selected from the group consisting of an anti-glaucoma agent, an anti-angiogenesis agent, an anti-infective agent, an anti-inflammatory agent, an analgesic agent, a local anesthetic, a growth factor, an immunosuppressant agent, an anti-allergic agent, an anti-oxidant, a cytokine, and combinations thereof. In some embodiments, the one or more diagnostic agents include paramagnetic molecules, fluorescent compounds, magnetic molecules, and radionuclides, x-ray imaging agents, contrast media.

In at least one embodiment, a method of treating, alleviating, and/or preventing an eye disorder is disclosed, the method comprising: administering to an eye of a subject an effective amount of a pharmaceutical composition comprising one or more types of mesenchymal stem cells (MSC), one or more types of MSC-derived exosomes, and/or one or more MSC-sourced compounds, thereby treating, alleviating, and/or preventing one or more symptoms of the eye disorder associated with the eye.

In at least another embodiment, the pharmaceutical composition alters one or more responses of one or more immune cells in the subject.

In at least a further embodiment, the one or more immune cells are selected from the group consisting of: dendritic cells, macrophages, T cells, natural killer (NK) cells, and combinations thereof.

In at least a further embodiment, the one or more MSC-sourced compounds are selected from the group consisting of: MSC-sourced IL-10, MSC-sourced TGF-β, MSC-sourced growth related oncogene (GRO), and combinations thereof.

In at least a further embodiment, the one or more MSC-sourced compounds (i) attenuate expression of one or more co-stimulatory molecules, and/or (ii) suppress production of one or more interleukins in macrophages and dendritic cells.

In at least a further embodiment, the one or more MSC-sourced compounds are selected from the group consisting of: MSC-sourced nitric oxide, MSC-sourced TGF-β, and prostaglandin E2 (PGE2), and combinations thereof.

In at least a further embodiment, the one or more MSC-sourced compounds (i) suppress proliferation and expansion of CD4+ Th1 cells, CD+ Th17 cells, and CD8+ cytotoxic T cells, (ii) decrease cytokine production, and/or (iii) decrease cytotoxicity.

In at least a further embodiment, the one or more MSC-sourced compounds comprise MSC-sourced indoleamine 2,3 dioxygenase (IDO), and the MSC-sourced IDO induces expansion of immunosuppressive Tregs.

In at least a further embodiment, the one or more MSC-sourced compounds comprise one or more microRNAs selected from the group consisting of: microRNA-10a-5p, microRNA-10b-5p, microRNA-191-5p, and combinations thereof.

In at least a further embodiment, the one or more microRNAs (i) prevent apoptosis, and/or (ii) facilitate viability of injured epithelial cells and limbal stem cells.

In at least a further embodiment, the one or more MSC-sourced compounds comprise microRNA-125b, and the microRNA-125b regulates antibody secretion in plasma cells in the eye.

In at least a further embodiment, the pharmaceutical composition is administered as a solution, a suspension, an ointment, a spray, drops, and/or a gel.

In at least an additional embodiment, the method further comprises administering to the subject one or more additional agents in combination with the pharmaceutical composition, and the one or more additional agents are selected from the group consisting of: an adjuvant, an antigen, an excipient, a vaccine, an allergen, an antibiotic, a gene therapy vector, a kinase inhibitor, a co-stimulatory molecule, a Toll-like receptor (TLR) agonist, a TLR antagonist, a therapeutic agent, a prophylactic agent, a diagnostic agent, an antimicrobial agent, an analgesic, a local anesthetic, an anti-inflammatory agent, an anti-oxidant agent, an immunosuppressant agent, an anti-allergenic agent, an enzyme cofactor, an essential nutrient, a growth factor, and combinations thereof.

In at least an additional embodiment, the administering to the eye further comprises administering, with the pharmaceutical composition, a pharmaceutically acceptable carrier.

In at least a further embodiment, the eye disorder is ocular graft-versus-host disease (oGVHD), and the one or more symptoms comprises dry eye.

In at least a further embodiment, the eye disorder is selected from the group consisting of: graft-versus-host disease (GVHD), acute GVHD, chronic GVHD, dry eye disease, ocular burns, tears or injury to the eye or associated structures, corneal neovascular disorders, corneal injuries, ocular blast injuries, eye infections, eye surgeries, drug-induced eye conditions, prolonged redness and inflammation of the eye, cataracts, age-related degeneration of the eye or vision associated therewith, amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection, autoimmune uveitis, infectious uveitis, anterior uveitis, posterior uveitis, pan-uveitis, inflammatory disease of the vitreous or retina, endophthalmitis, macular edema, macular degeneration, age related macular degeneration, proliferative and nonproliferative diabetic retinopathy, hypertensive retinopathy, autoimmune disease of the retina, primary and metastatic intraocular melanoma or other intraocular metastatic tumors, open angle glaucoma, closed angle glaucoma, pigmentary glaucoma, and combinations thereof.

In at least a further embodiment, the pharmaceutical composition is administered prior to, in conjunction with, subsequent to, or alternating with, one or more therapeutic, prophylactic, and/or diagnostic agents.

In at least a further embodiment, the one or more therapeutic, prophylactic, and/or diagnostic agents is selected from the group consisting of: an anti-glaucoma agent, an anti-angiogenesis agent, an anti-infective agent, an anti-inflammatory agent, an analgesic agent, a local anesthetic, a growth factor, an immunosuppressant agent, an anti-allergic agent, an anti-oxidant, a cytokine, and combinations thereof.

In at least one embodiment, a pharmaceutical composition is disclosed comprising: one or more types of mesenchymal stem cells (MSC), one or more types of MSC-derived exosomes, and/or one or more MSC-sourced compounds (e.g., microRNAs sourced from the one or more types of MSC-derived exosomes), and one or more pharmaceutically acceptable excipients.

In at least one embodiment, a kit is disclosed comprising: a container containing one or more single, sterile unit doses of the pharmaceutical composition.

In at least an additional embodiment, the pharmaceutical composition is in a pharmaceutically acceptable carrier for administration to an eye of a subject.

In at least one embodiment, a method of treating, alleviating, or preventing an eye disorder is disclosed, the method comprising: administering to the eye of a subject an effective amount of a pharmaceutical composition comprising one or more types of microRNAs, thereby treating, alleviating, and/or preventing one or more symptoms of the eye disorder associated with the eye.

In at least an additional embodiment, the one or more types of microRNAs are sourced from one or more mesenchymal stem cell (MSC)-derived exosomes.

Therefore, based on the foregoing and continuing description, the subject invention in its various embodiments may comprise one or more of the above-mentioned features in any non-mutually-exclusive combination.

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows various effects of immunoablative chemotherapy and irradiation, which are generally used as “precondition treatments” prior to allogeneic HSC transplantation and which are considered to be the main risk factors for the development of acute graft-versus-host disease (aGVHD).

FIG. 2 shows various effects of MSCs and/or MSC-based therapies when administered to manage and/or treat one or more eye disorders in a subject, according to at least one embodiment of the disclosure.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment,” “an embodiment,” “an example embodiment,” etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may affect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms “first,” “second,” etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, “at least one of A, B, and C” indicates A or B or C or any combination thereof. As used herein, the singular forms “a,” “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise,” “comprises,” and “comprising” are to be interpreted inclusively rather than exclusively. Likewise, the terms “include,” “including,” and “or” should all be construed to be inclusive, unless such a construction is clearly prohibited from the context. The terms “comprising” or “including” are intended to include embodiments encompassed by the terms “consisting essentially of” and “consisting of.” Similarly, the term “consisting essentially of” is intended to include embodiments encompassed by the term “consisting of.” Although having distinct meanings, the terms “comprising,” “having,” “containing,” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Terms such as, among others, “about,” “approximately,” “approaching,” or “substantially,” mean within an acceptable error for a particular value or numeric indication as determined by one of ordinary skill in the art, which depends in part on how the value is measured or determined. The aforementioned terms, when used with reference to a particular non-zero value or numeric indication, are intended to mean plus or minus 10% of that referenced numeric indication. As an example, the term “about 4” would include a range of 3.6 to 4.4. All numbers expressing dimensions, velocity, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise.

### Definitions

The following is a non-exhaustive and non-limiting list of terms used herein and their respective definitions.

The terms “agent” or “active agent,” which are used interchangeably herein, refer to a physiologically or pharmacologically active substance that acts locally and/or systemically in a subject's body. An “agent” or “active agent” is a compound or substance that is administered to an individual for the treatment (e.g., therapeutic agent, cancer therapeutic agent, and the like), prevention (e.g., prophylactic agent), or diagnosis (e.g., diagnostic agent) of a disease or disorder. Such agents may also include therapeutics that prevent or alleviate symptoms, such as, for instance, symptoms associated with one or more eye disorders or treatments for such disorders. “Ophthalmic drug” or “ophthalmic active agent,” as used herein, refers to an agent that is administered to a patient to alleviate, delay onset of, and/or prevent one or more symptoms of a disease or disorder of the eye, or a diagnostic agent useful for imaging or otherwise assessing the eye.

The term “administering” or “administration” refers to providing or giving a subject one or more agents and/or formulations, such as one or more d-MAPPS pharmaceutical compositions, either alone or in conjunction with any other compound and/or agent (including, e.g., prophylactic or therapeutic agents), by any effective route. Exemplary routes of administration include, but are not limited to, injection (such as, e.g., subcutaneous, subdermal, intramuscular, intradermal, intraperitoneal, intracerebroventricular, intraosseous, intratumoral, intraprostatic, and intravenous), transdermal, intranasal, oral, vaginal, rectal, and inhalation.

The term “amniotic factor” generally refers to one or more compounds naturally present in the amniotic fluid. These include, for example, carbohydrates, proteins and peptides (e.g., enzymes, hormones), lipids, metabolic substrates and products (e.g., lactate, pyruvate), and electrolytes.

The term “antigen” refers to a compound, composition, and/or substance that can stimulate the production of antibodies or an immune response in a subject, including compositions that are injected or absorbed into a subject. An “antigen” may react with the products of specific humoral and/or cellular immunity, including, for example, those induced by heterologous antigens.

The term “biocompatible” or “biologically compatible,” as used herein, generally refers to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient or subject.

The term “biodegradable polymer,” as used herein, generally refers to a polymer that will degrade or erode by enzymatic action and/or hydrolysis under physiologic conditions to smaller units or chemical species that are capable of being metabolized, eliminated, or excreted by the subject. The degradation time is a function of polymer composition, morphology, such as porosity, particle dimensions, and environment.

The term “cancer” refers to a class of diseases or conditions in which abnormal cells divide without control and can invade nearby tissues. A malignant cancer is one in which a group of tumor cells display one or more of uncontrolled growth (e.g., division beyond normal limits), invasion (e.g., intrusion on and destruction of adjacent tissues), and/or metastasis (e.g., spread to other locations in the subject's body via lymph or blood). As used herein, the terms “metastasis” or “metastasize” refer to the spread of cancer from one part of the body to another. A tumor formed by cells that have spread is called a “metastatic tumor” or a “metastasis.” The metastatic tumor contains cells that are similar to those in the original tumor (i.e., the tumor at the primary site of tumor growth). A “cancer cell” or “tumor cell” refers to an individual cell of a cancerous growth or tissue. A “tumor” refers generally to a swelling or lesion formed by an abnormal growth of cells, which may be benign, pre-malignant, or malignant. Most cancers form tumors, but some, e.g., leukemia, and some blood cancers, do not necessarily form tumors. For those cancers that form tumors, the terms “cancer,” “cancer cell,” “tumor,” and “tumor cell” are used interchangeably. The amount of a tumor in a given subject is the “tumor burden,” which can be measured as the number, volume, and/or weight of the tumor.

The term “combination therapy” refers to the administration of different compounds, agents, and/or individual therapies in a sequential and/or simultaneous manner. Individual elements of a “combination therapy” may be administered at different times and/or by different routes, but act in combination to provide a beneficial effect on the subject.

The term “compound” refers to a substance formed from one or more chemical elements, arranged together in any proportion or structural arrangement. The one or more chemical elements may be either naturally occurring and/or non-naturally occurring. As used herein, the term “biological compound” refers to a compound of biological origin and/or having one or more effects on a subject's local and/or systemic biological functions. Accordingly, “compounds” or “biological compounds” include, as non-limiting examples, various proteins (e.g., growth factors, hormones, enzymes), nucleic acids, and pharmaceutical products (e.g., drugs, prodrugs). The term “drug” generally refers to a medicine or other substance that has a physiological effect when introduced into a subject. The term “prodrug” generally refers to a biologically and/or chemically inactive compound that can be metabolized by a subject to produce a drug.

The terms “decrease,” “lower,” “lessen,” “reduce,” and “abate,” which are used interchangeably herein, refer generally to the ability of a compound, formulation, or therapy (including those disclosed herein) to produce, elicit, and/or cause a lesser physiological response (e.g., downstream effects) compared to the response caused by a respective control compound, formulation, or therapy. A “decrease” or “reduced” amount is typically a “statistically significant” amount, and may include a decrease that is, for instance, 1.1, 1.2, 1.5, 2, 3, 4, 5, 6, 7, 8, 9, 10, 15, 20, or 30 or more times (e.g., 500, 1000 times) (including all integers and decimal points in between and above 1, e.g., 1.5, 1.6, 1.7. 1.8, etc.).

The term “dendritic cell” refers to a type of specialized antigen-presenting cell (“APC”) involved in innate and/or adaptive immunity. Dendritic cells may also be referred to herein as “DC” or “DCs.” Dendritic cells may be present in the tumor microenvironment, and these are referred to as “tumor-associated dendritic cells” (“tDC” or “tDCs”).

The terms “effective amount” or “therapeutically effective amount,” which are used interchangeably herein, refer to the amount of an agent (e.g., including one or more d-MAPPS pharmaceutical compositions described herein) that is sufficient to effect beneficial or desired therapeutic result, including clinical results. An “effective amount” may vary depending upon one or more of: the subject and disease condition being treated, the sex, weight and age of the subject, the severity of the disease condition, the manner of administration, the ability of one or more formulations to elicit a desired response in the subject, and the like. The beneficial therapeutic effect can include, but is not limited to, enablement of diagnostic determinations; prevention of disease or tumor formation; amelioration of a disease, symptom, disorder, and/or pathological condition; reducing or preventing the onset of a disease, symptom, disorder, and/or pathological condition; and generally counteracting a disease, symptom, disorder, and/or pathological condition. The term “effective amount” includes an amount that is effective to “treat” a subject (e.g., a patient or individual), including an amount effective to alleviate, delay onset of, and/or prevent one or more symptoms, particularly of a disease or disorder of the eye. When a therapeutic amount is indicated, the precise amount of one or more formulations described in the present disclosure to be administered can be determined by a physician, based on, for instance, considerations such as individual differences in age, weight, extent of the disease or disorder, and/or condition of the subject (individual).

The terms “enhance,” “induce,” “induction,” and “increase,” which are used interchangeably herein, refer generally to the ability of a compound, formulation, or therapy (including those disclosed herein) to produce, elicit, and/or cause a greater physiological response (e.g., downstream effects) compared to the response caused by a respective control compound, formulation, or therapy. An “enhanced” or “increased” amount is typically a “statistically significant” amount, and may include an increase that is, for instance, 1.1, 1.2, 1.5, 2, 3, 4, 5, 6, 7, 8, 9, 10, 15, 20, or 30 or more times (e.g., 500, 1000 times) (including all integers and decimal points in between and above 1, e.g., 1.5, 1.6, 1.7. 1.8, etc.).

The term “growth factor” refers to any compound (e.g., one or more groups of proteins or hormones) that stimulate cellular growth. Generally, growth factors play an important role in promoting cellular differentiation and cell division, and they occur in a wide range of organisms, including humans.

The term “immune cell” refers to any cell of the immune system that has one or more effector functions (e.g., cytotoxic cell killing activity, secretion of cytokines, induction of antibody-dependent cell-mediated cytotoxicity (ADCC), and/or induction of complement-dependent cytotoxicity (CDC)).

The terms “immunologic,” “immunological,” or “immune” response, which are used interchangeably herein, refer to the development of a beneficial humoral (i.e., antibody-mediated) and/or a cellular (e.g., mediated by immune cells, such as antigen-specific T cells, or their secretion products) response directed against an antigen and/or immunogen in a specific subject. Such a response can be an active response induced by administration of an antigen and/or immunogen, or a passive response induced by administration of antibodies or primed T-cells. A cellular immune response is elicited by the presentation of polypeptide epitopes in association with Class I or Class II major histocompatibility complex (MHC) molecules to activate antigen-specific CD4+ healer T cells and/or cos+ cytotoxic T cells. The response may also involve, for instance, activation of monocytes, macrophages, natural killer (NK) cells, basophils, dendritic cells, astrocytes, microglia cells, eosinophils, and/or other components of innate immunity. The presence of a cell-mediated immunological response can be determined by proliferation assays (e.g., CD4+ T cells) or cytotoxic T lymphocyte (CTL) assays. The relative contributions of humoral and cellular responses to the protective or therapeutic effect of an antigen and/or immunogen can be distinguished by, for example, separately isolating antibodies and T cells from an immunized syngeneic animal and measuring the protective or therapeutic effect in a second subject.

The term “implant,” as generally used herein, refers to a polymeric device or element that is structured, sized, or otherwise configured to be implanted, preferably by injection or surgical implantation, in a specific region of the body so as to provide therapeutic benefit by releasing one or more therapeutic, prophylactic or diagnostic agents over an extended period of time at the site of implantation. For example, intraocular implants are polymeric devices or elements that are structured, sized, or otherwise configured to be placed in the eye, preferably by injection or surgical implantation, and to treat one or more diseases or disorders of the eye by releasing one or more therapeutic, prophylactic or diagnostic agents over an extended period. Intraocular implants are generally biocompatible with physiological conditions of an eye and do not cause adverse side effects. Generally, intraocular implants may be placed in an eye without disrupting vision of the eye.

The term “ionizing radiation” refers to radiation, traveling as a particle or electromagnetic wave, that carries sufficient energy to detach electrons from atoms or molecules, thereby ionizing an atom or a molecule. Generally, ionizing radiation is made up of energetic subatomic particles, ions, or atoms moving at high speeds and electromagnetic waves on the high-energy end of the electromagnetic spectrum. Radiation has been demonstrated to induce adaptive immune responses to mediate tumor regression. In addition, the induction of type I interferons (“IFNs”) by radiation is essential for the function of CD8+ T cells. Radiation induces cell stress and causes excess deoxyribonucleic acid (DNA) breaks, indicating that the nucleic acid-sensing pathway likely accounts for the induction of type I IFNs upon radiation. Type I IFN responses in DCs dictate the efficacy of antitumor radiation. In contrast, chemotherapeutic agents and anti-human epidermal growth factor receptor 2 (HER2) antibody treatments have been demonstrated to depend on a distinct immune mechanism to trigger adaptive immune responses. In general, therapeutic radiation-mediated antitumor immunity depends on a proper cytosolic DNA sensing pathway. In at least one embodiment, one or more agents, formulations, and/or methods (e.g., including one or more types of MSCs, either alone or in conjunction with one or more other agents) described herein is administered in combination with radiation therapy.

The term “macrophage” refers to a type of white blood cell of the immune system that engulfs and digests cellular debris, foreign substances, microbes, cancer cells, and the like. These phagocytes include various subtypes (e.g., histiocytes, Kupffer cells, alveolar macrophages, microglia, and others), but all are part of the mononuclear phagocyte system. Besides phagocytosis, macrophages play a critical role in both innate and adaptive immunity by recruiting other endogenous immune cells (e.g., lymphocytes). For example, they are important as antigen presenters to T cells. In humans, dysfunctional macrophages can cause severe diseases (e.g., chronic granulomatous disease) that result in frequent infections. Beyond increasing inflammation and stimulating the immune system, macrophages also play an important anti-inflammatory role and can decrease immune reactions through the release of various compounds (e.g., cytokines). Macrophages that encourage inflammation may be termed “M1 macrophages” because they have the so-called “M1 phenotype,” whereas those that decrease inflammation and encourage tissue repair may be termed “M2 macrophages” because they have the so-called “M2 phenotype.”

The term “mean particle size,” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

The term “microparticle,” as used herein, generally refers to a particle having a diameter, such as an average diameter, from about 1 micron to about 100 microns, preferably from about 1 micron to about 50 microns, more preferably from about 1 to about 30 microns. The microparticles can have any shape. Microparticles having a spherical shape are generally referred to as “microspheres.”

The term “molecular weight” generally refers to the relative average chain length of a bulk polymer or protein, unless otherwise specified. In practice, molecular weights can be estimated or characterized using various methods including, for example, gel permeation chromatography (GPC) or capillary viscometry. GPC molecular weights are reported as the weight-average molecular weight (MW), as opposed to the number-average molecular weight (MN). Capillary viscometry provides estimates of molecular weight as the inherent viscosity determined from a dilute polymer solution using a particular set of concentration, temperature, and solvent conditions.

The term “nanoparticle,” as used herein, generally refers to a particle having a diameter, such as an average diameter, from about 10 nanometers (nm) up to but not including about 1 micron, preferably from 100 nm to about 1 micron. The particles can have any shape. Nanoparticles having a spherical shape are generally referred to as “nanospheres.”

The term “parenteral administration” refers to a type of administration by any method other than through the digestive tract or non-invasive topical or regional routes. As a non-limiting example, parenteral administration may include administration to a subject via intravenous, intradermal, intraperitoneal, intrapleural, intratracheal, intraarticular, intrathecal, intramuscular, subcutaneous, subjunctival, injection, and/or infusion.

The term “peptide” refers to a polymer of amino acid residues. The amino acid residues may be naturally occurring and/or non-naturally occurring. The terms “polypeptide,” “peptide,” and “protein” are used interchangeably herein. The terms apply to, for instance, amino acid polymers of one or more amino acid residues, an artificial chemical mimetic of a corresponding naturally occurring amino acid, naturally occurring amino acid polymers, and non-naturally occurring amino acid polymers.

The term “pharmaceutically acceptable,” as used herein, refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “subject,” “individual,” or “patient,” which are used interchangeably herein, refer to a vertebrate, such as a mammal (e.g., a human). Mammals include, but are not limited to, murines (e.g., mice), simians, humans, farm animals, sport animals, and pets. In at least one embodiment, the subject is a non-human mammal, such as a monkey or other non-human primate, mouse, rat, rabbit, guinea pig, pig, goat, sheep, dog, cat, horse, or cow. In at least one example, the subject has a tumor, such as a cancer, that can be treated using one or more agents, formulations, and/or methods (e.g., including one or more d-MAPPS pharmaceutical compositions, either alone or in conjunction with one or more other agents) disclosed herein. In at least an additional example, the subject is a laboratory animal/organism, such as, for example, a mouse, rabbit, guinea pig, or rat. In at least a further example, a subject includes, for instance, farm animals, domestic animals and/or pets (e.g., cats, dogs). In at least a still further example, a subject is a human patient that has one or more eye disorders, has been diagnosed with an eye disorder, and/or is at risk of having an eye disorder. A “patient” can specifically refer to a subject that has been diagnosed with a particular disease, condition, and/or indication that can be treated with refers to a subject that has been diagnosed with a particular indication that can be treated with one or more agents, formulations, and/or methods (e.g., including one or more d-MAPPS pharmaceutical compositions, either alone or in conjunction with one or more other agents) disclosed herein.

The term “topical administration” refers to a type of non-invasive administration to the skin, orifices, and/or mucosa of a subject. Topical administrations can be administered locally; that is, they are capable of providing a local effect in the region of application without systemic exposure. Topical formulations can, however, provide one or more systemic effects via, e.g., adsorption into the blood stream of the individual. Routes of topical administration include, but are not limited to, cutaneous and transdermal administration, buccal administration, intranasal administration, intravaginal administration, intravesical administration, ophthalmic administration, pulmonary administration, and rectal administration.

The terms “treating,” “treatment,” and “therapy” refer, either individually or in any combination, to any success or indicia of success in the attenuation or amelioration of an injury, disease, symptom, disorder, pathology, and/or condition, and/or pathological condition, including any objective or subjective parameter such as, for instance, abatement, remission, diminishing of symptoms or making the condition more tolerable to the patient, slowing the rate of degeneration or decline, making the final point of degeneration less debilitating, improving a subject's physical or mental well-being, and/or prolonging the length of survival. Treatment does not necessarily indicate complete eradication or cure of the injury, disease, symptom, disorder, pathology, and/or condition, and/or pathological condition, or any associated symptom(s) thereof. The treatment may be assessed by one or more objective or subjective parameters, including, for example, the results of a physical examination, blood and other clinical tests (e.g., imaging), and the like. In at least one example, treatment with the disclosed one or more agents, formulations, and/or methods (e.g., including one or more d-MAPPS pharmaceutical compositions, either alone or in conjunction with one or more other agents) results in a clinical improvement in one or more eye diseases in a subject.

Further, unless otherwise noted, technical terms are generally used according to conventional usage. Aspects of the disclosed methods employ, unless indicated specifically to the contrary, conventional methods of chemistry, biochemistry, organic chemistry, molecular biology, microbiology, recombinant DNA techniques, genetics, immunology, and/or cell biology, many of which are described below solely for the purpose of illustration. Such techniques are explained fully in technical literature sources. General definitions of common terms in the aforementioned fields, including, for instance, molecular biology, may be found in references such as, e.g., Krebs et al., Lewin's Genes X, Jones & Bartlett Learning (2009) (ISBN 0763766321); Redei, Encyclopedic Dictionary of Genetics, Genomics, Proteomics and Informatics (3rd ed.), Springer (2008) (ISBN: 1402067532); Ausubel et al., Current Protocols in Molecular Biology, John Wiley and Sons (updated July 2008) (ISBN: 047150338X); Ausubel et al., Short Protocols in Molecular Biology: A Compendium of Methods from Current Protocols in Molecular Biology (2nd ed.), Wiley-Interscience (1989) (ISBN 0471514705); Glover, et al., DNA Cloning: A Practical Approach, Vol. I-II, Oxford University Press (1985) (ISBN 0199634777); Anand et al., Techniques for the Analysis of Complex Genomes, Academic Press (1992) (ISBN 0120576201); Hames et al., Transcription and Translation: A Practical Approach, Oxford University Press (1984) (ISBN 0904147525); Perbal et al., A Practical Guide to Molecular Cloning (2nd ed.), Wiley-Interscience (1988) (ISBN 0471850713); Kendrew et al., Encyclopedia of Molecular Biology, Wiley-Blackwall (1994) (ISBN 0632021829); Meyers et al., Molecular Biology and Biotechnology: A Comprehensive Desk Reference, Wiley-VCH (1996) (ISBN 047118571X); Harlow et al., Antibodies: A Laboratory Manual, Cold Spring Harbor Laboratory Press (1988) (ISBN 0879693746); Coligan et al., Current Protocols in Immunology, Current Protocols (2002) (ISBN 0471522767); Annual Review of Immunology; articles and/or monographs in scientific journals (e.g., Advances in Immunology); and other similar references.

### Graft-Versus-Host Disease and Ocular Graft-Versus-Host Disease

Graft-versus-host disease (GVHD) is a severe, systemic disorder which develops as a serious and life-threatening complication of allogeneic hematopoietic stem cell (HSC) transplantation. GVHD predominantly occurs in the skin, gastrointestinal tract, liver, oral mucosa, and in the eyes of patients as a result of immune dysregulation and inflammation-mediated tissue destruction that leads to fibrosis and organ dysfunction. The acute form of GVHD (aGVHD) is generally diagnosed if pathological events develop within 100 days after HSC transplantation, while the diagnosis of chronic GVHD disease (cGVHD) is generally made when GVHD-related clinical signs are observed 100 days after HSC transplantation.

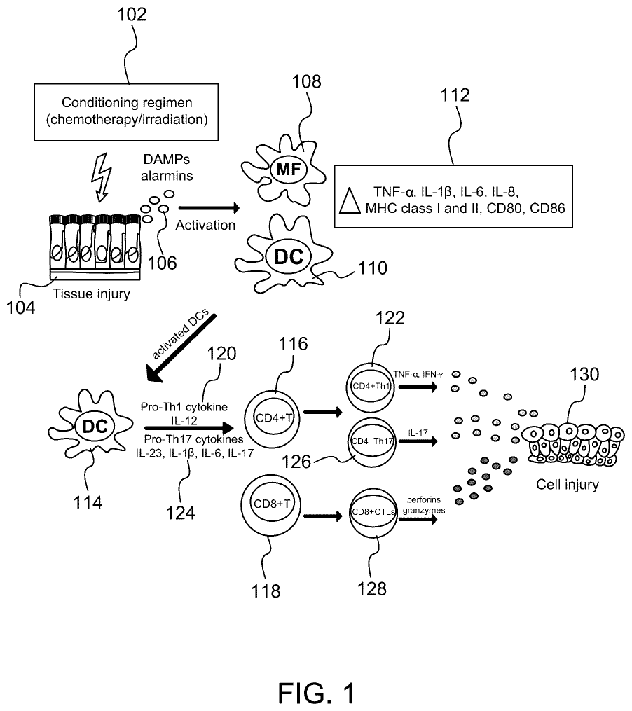

Immunoablative chemotherapy and irradiation, which are generally used as “precondition treatments” prior to allogeneic HSC transplantation, are considered as the main risk factors for the development of aGVHD, as shown in FIG. 1. Specifically, chemotherapy and/or irradiation 102, which are used as “precondition treatment” prior to allogeneic HSC transplantation, cause tissue injury 104. This results in the release 106 of damage-associated molecular patterns (“DAMPs”) and alarmins (e.g., heat-shock proteins, interleukin (IL)-33) from injured parenchymal cells, which, in turn, activate tissue resident macrophages (denoted “MF”) 108 and dendritic cells (DCs, denoted “DC”) 110. Such activation results in effects 112, specifically, production of a large amount of inflammatory cytokines (e.g., tumor necrosis factor alpha (TNF-α), IL-1 beta (IL-1β), IL-6, IL-8), and an increased expression of major histocompatibility complex (MHC) and co-stimulatory molecules (CD80, CD86) on their membranes. Activated DCs 114 capture antigens from damaged cells and bring them into the regional lymph nodes to activate donor cluster of differentiation (CD)4+ T helper 116 and CD8+ cytotoxic T cells 118. Inflammatory, IFN-γ-producing CD4+ Th1 and IL-17-producing CD4+ Th17 lymphocytes are considered as the main effector cells in the pathogenesis of aGVHD. DC-derived IL-12 120 is crucially responsible for the development of Th1 cells 122, while DC-sourced IL-1β, IL-6, and IL-23 (collectively 124) can induce generation of effector Th17 cells 126. In addition to CD4+ T helper cells, perforin, and granzyme B-producing CD8+ cytotoxic T cells (CTLs) 128 and natural killer (NK) cells are also involved in tissue destruction during the progression of aGVHD. Long-term tissue destruction 130 driven by Th1, Th17, CTLs, and NK cells can result in the development of cGVHD. IFN-γ and IL-17 derived from Th1 and Th17 cells activate tissue resident macrophages and circulating neutrophils which, in turn, produce matrix metalloproteinases (MMPs), inflammatory cytokines, and pro-fibrotic transforming growth factor beta (TGF-β), resulting in fibrosis and organ dysfunction.

T-cell-recruiting chemokines (e.g., CXCL3, CXCL9, and CXCL11) and inflammatory cytokines (e.g., TNF-α, IL-1β, IL-6, IFN-γ) are highly elevated in the tears, and at the ocular surface, of patients suffering from GVHD. Conjunctiva-associated mucosal tissue mimics systemic mucosal membranes of the lungs, intestines, and mouth, making it an ideal target for activated inflammatory cells. Accordingly, although all components of the ocular surface (e.g., cornea, conjunctiva, eyelids, lacrimal glands, meibomian glands, and lacrimal drainage system) may be injured during the progression of ocular GVHD (oGVHD), keratoconjunctivitis sicca (dry eye disease (DED)) is the most frequent clinical sign of oGVHD. It usually develops 6 to 12 months after HSC transplantation and is observed in majority (e.g., up to 70%) of patients suffering from cGVHD. Th1 and Th17 cell driven inflammation, as well as CTL and NK cell mediated injury of corneal epithelial cells and meibomian glands, can result in corneal epitheliopathy, corneal ulceration, and meibomian gland dysfunction (MGD). The loss of homeostasis, hyperosmolarity of the tears, and persistent intraocular inflammation can lead to neurosensory dysfunction and/or visual disturbance. Consequently, dryness, burning or scratchy sensations in eyes, and foreign body sensations are frequently reported by patients with oGVHD. Severe inflammation may cause punctate epithelial keratopathy and painful epithelial erosions which can be complicated with secondary infections, stromal necrosis, limbal stem cell deficiency (LSCD), and corneal perforation.

In addition to DED, other conditions (e.g., uveitis, glaucoma, posterior scleritis, optic nerve edema, retinal detachment, and chorioretinopathy) can also be observed in patients suffering from cGVHD. Such conditions may develop due to the detrimental immune response driven by donor T lymphocytes or represent severe complications of immunosuppressive therapy used for the treatment of systemic GVHD.

A three-pronged treatment approach is often used as a standard therapy for the treatment of oGVHD. Lubrication and tear preservation with topical administration of non-preserved phosphate-free artificial tears is the first-line treatment approach. Frequent use of tear substitutes preserves the ocular surface, reduces concentration of inflammatory mediators at the ocular surface, and prevents aggravation of ongoing inflammation. Warm compresses, lid scrubs, and maintenance of lid hygiene are often used as a second-line treatment approach, which should prevent tear evaporation and tear film instability. Finally, considering the important role of detrimental immune responses in oGVHD development and progression, the attenuation of ocular inflammation represents the third and most important step in the therapy of oGVHD. For this purpose, topical administration of corticosteroids (e.g., methylprednisolone, prednisone) and immunosuppressive drugs (e.g., Cyclosporin (CsA), Tacrolimus) have been used. Aggressive topical steroid therapy accompanied with pseudomembrane removal can significantly enhance epithelial healing and suppress fibrosis in the eyes of oGVHD patients with pseudomembranous conjunctivitis. Topical administration of steroids should not be used for the treatment of oGVHD patients with corneal epithelial defects, stromal thinning, or eye infections. Topical CsA and Tacrolimus eye drops have been used for the treatment of patients who suffered from mild-to-severe chronic oGVHD with DED refractory to lubrication and steroid therapy.

In addition to lubrication and immunosuppression, repair and regeneration of injured corneal epithelium and restoration of meibomian and lacrimal glands are also important therapeutic approaches in oGVHD treatment. For this purpose, administration of autologous serum which contains epithelial growth factors, cytokines, nerve growth factor (NGF), vitamin A, fibronectin, and TGF-β, can be used. The contraindications for the use of autologous serum are the presence of active ocular or systemic inflammation, local or systemic bacterial and viral infections, and poor general health. Additionally, due to the strict legal regulations for the use of blood products and transfusions, topical administration of autologous serum eye drops are limited to specialized medical centers.

TABLE 1
Main purposes and weaknesses of currently used therapeutic agents
in oGVHD treatment and advantages of MSCs/MSC-Exos-based therapy

| | | | Advantages of |
|---|---|---|---|
| | | | MSCs/MSC-Exos- |
| Therapeutic Agent | Purpose | Weakness(es) | based Therapy |
| artificial | lubrication and | limited ocular penetration; | MSC-Exos, due to |
| tears | tear preservation | could not suppress | nano-sized dimension |
| | | inflammatory immune | and lipid envelope, |
| | | cells in the eyes; | can avoid all |
| | | could not enhance | biological barriers |
| | | viability of epithelial | in the eyes and can |
| | | cells | deliver their cargo |
| | | | directly into the |
| | | | parenchymal and |
| | | | immune cells |
| corticosteroid- | suppression of eye | could not promote viability | MSCs could differentiate |
| containing eye | inflammation | of injured cells; | in epithelial cells and |
| drops | | long-term use could | may deliver trophic |
| | | result in the development | factors in injured cells |
| | | glaucoma, cataract and | enhancing their viability. |
| | | corneal thinning | Long-term use of MSC- |
| | | | Exos could efficiently |
| | | | suppress intraocular |
| | | | inflammation without |
| | | | causing severe side effects |
| immunosuppressive | inhibition of detrimental | could not suppress | MSCs could enhance |
| eye drops | immune response in the | cell-death associated | viability and may |
| | eyes of oGVHD patients | signaling pathway | inhibit apoptosis of |
| | | in damaged cells; long- | injured cells in |
| | | term use could result | corneal tissue, |
| | | in the development of | meibomian and lacrimal |
| | | secondary immunodeficiency | glands. Long-term use |
| | | and infectious keratitis | of locally transplanted |
| | | | MSCs and MSC-Exos could |
| | | | efficiently inhibit eye |
| | | | inflammation without |
| | | | causing secondary infection |

As can be seen in Table 1, none of the currently used therapeutic agents were able to efficiently suppress ongoing eye inflammation and failed to completely prevent immune cell-driven injury of epithelial cells in the eyes of oGVHD patients. For instance, the bioavailability of immunoregulatory eye drops is generally low since the well-developed protective mechanisms of the eye ensure their rapid clearance from the pre-corneal space, limiting ocular penetration and therapeutic efficacy of the incorporated drugs. Eye drops which are used in the treatment of oGVHD do not contain growth factors which are able to promote viability and suppress cell-death associated signaling pathway in damaged cells. Therefore, none of the currently available therapeutic agents provide trophic support to the injured cells or promote repair and regeneration of injured corneal, meibomia, and lacrimal epithelial cells in the eyes of oGVHD patients. Additionally, severe side effects are possible after prolonged use of corticosteroids and immunosuppressive eye drops. For instance, long-term steroid use can result in the development of glaucoma, cataract, and corneal thinning in the eyes of oGVHD patients. Similarly, prolonged use of immunosuppressive drugs can lead to the development of severe immunodeficiency, significantly increasing the risk for the development of secondary infectious keratitis. The existing inadequacies in oGVHD treatment imply that there is an urgent need for the clinical use of new therapeutic agents which will suppress on-going eye inflammation without causing severe immunosuppression and will, at the same time, promote repair and regeneration of injured epithelial cells in the eyes of oGVHD patients.

### Mesenchymal Stem Cells (MSCs) and MSC-Mediated Tissue Repair and Regeneration

Mesenchymal stem cells (“MSC” or “MSCs”) are self-renewable adult stem cells which are able to differentiate into corneal epithelial cells under specific culture conditions. Additionally, MSC secrete a large number of growth factors that support the viability of injured cells and produce immunomodulatory proteins which regulate the phenotype and/or function of immune cells that participate in the development and progression of oGVHD. Many MSC-derived bioactive factors are contained in MSC-sourced exosomes (MSC-Exos), extracellular vesicles which, due to their nano-sized dimension and lipid envelope, can easily bypass all biological barriers to reach the target epithelial and/or immune cells in the eyes and lacrimal system of oGVHD patients without affecting neighboring parenchymal cells and, therefore, without causing any severe side effects. Due to their enormous differentiation potential and immunosuppressive characteristics, MSC and MSC-Exos are new remedies in regenerative ophthalmology. Accordingly, in at least one embodiment of the present disclosure, d-MAPPS pharmaceutical compositions, including d-MAPPS solutions, include one or more MSCs and/or one or more MSC-Exos, as set forth in further detail herein.

MSC may, under specific culture conditions, differentiate in the cells of all three germ layers. Multi-lineage differentiation potential of MSCs could be a consequence of their complex development origin. During embryogenesis, different subpopulations of MSCs originate from different precursor cells, including epithelial-to-mesenchymal transition-derived cells, Sox1+ neuroepithelial cells, lateral plate mesoderm-derived mesoangioblast cells from the embryonic dorsal aorta, and blood-vessel-derived precursor cells.

MSC reside in almost all postnatal tissues from where MSC can be isolated, propagated in vitro, and used in cell-based therapies of degenerative and inflammatory diseases. For clinical use, MSCs can be most frequently derived from bone marrow (BM), umbilical cord (UC), amniotic fluid (AF), and adipose tissue (AT). Specific functional properties of BM-derived MSC (“BM-MSC” or “BM-MSCs”) which favor their clinical application include, for instance, rapid proliferation in vitro, genomic stability after long-term cultivation, and the capacity for the increased production of immunosuppressive cytokines. Although BM-MSC have enormous therapeutic potential, harvesting of BM is an invasive procedure and, therefore, UC, AF, and AT can be used as alternative tissue sources for the isolation of MSCs. Collection of UC-derived MSC (“UC-MSC” or “UC-MSCs”) is noninvasive, painless, and safe. UC-MSC share similar functional properties with BM-MSC, but have a higher capacity for exosome (Exos) production. AF, obtained through amniocentesis, serves as an important source of AF-derived MSC (“AF-MSC” or “AF-MSCs”). AF-MSCs can produce large amount of neurotrophins and have a high therapeutic potential in the repair and regeneration of injured neural cells. Lastly, AT-derived MSC (“AT-MSC” or “AT-MSCs”), easily derived from patients' AT, are usually used for autologous transplantation of MSC. AT-MSC have a high proliferation capacity and potent immunoregulatory properties.

Under specific culture conditions, BM-MSC and AT-MSC may differentiate into corneal epithelial cells. For instance, one week of exposure to hormonal epidermal medium (SHEM) or standard MSC cultured Dulbecco's Modified Eagle Medium (DMEM) supplemented with all-trans-retinoic acid (ATRA), may be sufficient to cause both BM-MSC and AT-MSC to differentiate into corneal epithelial cells.

Higher expression of epithelial markers (e.g., cytokeratin (CK)12, CK3, CK19, E-cadherin) and lower expression of mesenchymal markers (e.g., Vim, snail and alpha smooth muscle actin (α-SMA)) can occur in BM-MSC and AT-MSC which were cultured in SHEM (MSC^{SHEM}) or ATRA-supplemented medium (MSC^{ATRA}) than in BM-MSC and AT-MSC that grew under standard culture conditions (MSC^{DMEM}). Down-regulation or suppression of the Wnt/β-catenin signaling pathway is crucially responsible for BM-MSC and AT-MSC differentiation towards corneal epithelial cells. Importantly, human corneal epithelial cells (HCE) that were co-cultured with MSC^{SHEM} or MSC^{ATRA} can have an increased proliferation rate and an improved capacity for wound healing than HCE which grew with MSC^{DMEM}. The fact that MSC^{SHEM} or MSC^{ATRA} may better guide HCE-driven wound healing than MSC^{DMEM} indicates that SHEM or ATRA not only increases expression of pro-epithelial genes in MSC, but can also induce enhanced secretion of MSC-derived bioactive factors, which improve the viability and proliferation rate of injured HCE. From 720 different proteins which were detected in BM-MSC and AT-MSC-sourced secretome, around 122 proteins participate in the proliferation and differentiation of corneal epithelial cells. Specific proteins such as, for instance, TGF-β receptor type-1, TGF-β receptor type-2, Ras-related C3 botulinum toxin substrate 1, and/or Ras-related C3 botulinum toxin substrate 2 derived from UC-MSC can be responsible for MSC-mediated regulation of epithelial cell proliferation. These molecules activate Jun-N-terminal kinase (JNK) and p38 mitogen activated kinase in HCE, which can elicit signaling pathways that improve their proliferation and migration, which may contribute to the enhanced healing of corneal wounds.

MSC also may have a capacity to repair and regenerate injured corneal epithelium, meibomian and lacrimal glands, indicating their therapeutic potential in the treatment of oGVHD.

For instance, in a rabbit model of alkaline-induced corneal injury, human BM-MSC can differentiate into corneal epithelial cells and migrate into damaged corneal stroma, which can cause improved survival of corneal stromal cells, resulting in corneal regeneration and attenuation of alkaline-induced corneal damage. AT-MSC may also have therapeutic potential in the treatment of LSCD. For instance, human AT-MSC cultured on fibrin gel and grafted onto the damaged corneal surface of mice may cause the re-population of limbal stem cells and the regeneration of injured corneal epithelium. The effectiveness of MSC in LSCD treatment has also been shown in clinical settings. BM-MSC can be successfully engrafted in the eyes of patients suffering from LSCD, significantly improving corneal epithelial failure.

Additionally, rat BM-MSC has therapeutic potential in the regeneration of meibomian glands and in the restoration of meibomian gland function, at least in the context of benzalkonium chloride (BAC)-induced eye injury in rats as an animal model of DED. Reduction in microvilli at apical portions of corneal epithelium, vascular congestion in meiboian glands, large number of apoptotic cells, decreased number of goblet cells, reduced presence of secretory granules and massive leukocyte infiltration can occur in the eyes of BAC+ saline-treated rats. Topically applied MSC can engraft into the injured meibomian glands and in the damaged conjunctival epithelium, resulting in suppressed detrimental immune response and induced repair and regeneration of injured tissue. Meibomian glands may therefore have normal architecture, a significantly increased number of goblet cells with numerous secretory granules, only a paucity of lymphocytes and neutrophils, and few apoptotic cells were detected in the corneas, conjuctivas, and meibomian glands of BAC+BM-MSC-treated rats. Additionally, mean aqueous tear volume can significantly increase one week after MSC application, suggesting therapeutic efficacy of BM-MSC in the treatment of MGD and DED.

Murine models of aqueous-deficient dry eye disease (ADDED) have shown that, in addition to the restoration of meibomian gland structure, murine lacrimal gland-derived MSC (“LG-MSC” or “LG-MSCs”) can manage to efficiently regenerate injured lacrimal glands as well. ADDED can be induced by the ligation of the lacrimal duct. Duct ligation can then be removed (e.g., three days later), and MSC or saline can be injected into the lacrimal gland. Duct ligation can induce interstitial edema and massive injury of lacrimal glands. Consequently, acinar cells, which produce and secrete the primary tear fluid, may be shrunken and dysfunctional in ADDED mice. Immediately after their injection, LG-MSC can engraft in the stroma of lacrimal glands, adjacent to acinar structures. Weeks after removal of duct ligation, LG-MSC can recover vital acinar structures to, e.g., 62% of total lacrimal gland tissue, which is an increase of, e.g., 25% compared to spontaneous regeneration after saline injection. Tightly arranged acini, organized in lobules and surrounded by connective tissue, may be observed in MSC-treated lacrimal glands, but not in saline-treated lacrimal glands. A higher presence of proliferating, Ki67-positive cells and enhanced expression of MIST1 expression (acinus specific transcription factor) can also be observed in the LG-MSC-treated lacrimal glands, confirming LG-MSC-mediated restoration of acinar cells. Further, significantly reduced expression of caspase-3 in LG-MSC-treated lacrimal glands can indicate that LG-MSC suppressed the apoptosis of acinar cells. As a result of LG-MSC-mediated regeneration of lacrimal glands, the amounts of secreted tears in the eyes of MSC-treated ADDED animals may be, 21 days after MSC injection, similar to the baseline value which were measured at the ocular surface of healthy animals. MSC-dependent suppression of detrimental immune response may also be responsible for the beneficial effects of LG-MSC in the repair and regeneration of lacrimal glands. A significantly reduced number of Ly6G-expressing neutrophils and a lower number of CD68-expressing macrophages may be observed in MSC-treated lacrimal glands, 21 days after LG-MSC transplantation. Additionally, LG-MSC can down-regulate the synthesis of TNF-α in lacrimal gland-infiltrated immune cells and suppress TNF-α-driven injury of acinar cells, significantly contributing to improved tear secretion, suggesting that the immunomodulatory potential of MSC can be important for these beneficial effects in oGVHD treatment.

Treatment of oGVHD Via MSC-Dependent Suppression of Detrimental Immune Response in the Eyes

MSC from all tissue sources are potent immunoregulatory cells that produce a large number of immunomodulatory factors (e.g., IL-10, TGF-β, growth related oncogene (GRO), indoleamine 2,3 dioxygenase (IDO), nitric oxide (NO), interleukin 1 receptor antagonist (IL-1Ra), prostaglandin E2 (PGE2)), which can alter the phenotype and/or function of all immune cells that play a pathogenic role in the development and progression of oGVHD.

Table 2 below lists various MSC-sourced immunomodulatory factors, their respective target cell(s), their respective mechanism(s) of action, and their respective immunomodulatory effect(s).

TABLE 2
Molecular mechanisms responsible for MSC-based
immunoregulation in the treatment of oGVHD

| MSC-sourced | | Mechanism | Immunomodulatory |
|---|---|---|---|
| Factor | Target Cell | of Action | Effect |
| TGF-β | Inflammatory | inhibition of Jak/Stat | suppressed proliferation |
| | Th1 and Th17 | signaling pathway; G1 | of Th1 and Th17 cells |
| | cells | cell cycle arrest | |
| IDO | T regulatory | modulation of T cell | suppressed conversion of |
| | cells | receptor signaling | T regulatory cells into |
| | | | Th17 cells |
| NO | MSC | enhanced IDO activity | expansion of T regulatory |
| | | | cells |
| PGE2 | naïve T | inhibition of IL-2 | suppressed expansion |
| | cells | production | of activated T cells |
| PGE2, TGF-β | DC | inhibition of DC | down-regulated expression |
| | | maturation; expansion | of costimulatory molecules; |
| | | of tolerogenic DC | reduced antigen-presentation; |
| | | | decreased production of |
| | | | pro-Th1 and pro-Th17 |
| | | | cytokines; attenuated |
| | | | activation of naïve T cells |
| PGE2, TGF-β, | macrophages | induction of alternative | increased production of |
| IL-10 | | phenotype | immunosuppressive cytokines |
| TGF-β, NO | NK cells | G1 cell cycle arrest | inhibited proliferation |
| | | | of NK cells |
| IDO, PGE2 | NK cells | induction of regulatory | expansion of NK regulatory |
| | | phenotype | cells |
| IL-10 | NK cells | suppressed production of | attenuated cytotoxicity |
| | | perforins, granzymes, | of NK cells |
| | | inflammatory and | |
| | | cytotoxic cytokines | |

By suppressing the Jak-Stat signaling pathway in T cells, MSC-sourced TGF-β can induce G1 cell cycle arrest and prevent the proliferation of these cells. MSC-derived IDO can promote expansion of immunosuppressive T regulatory cells (Tregs) and prevent their conversion in inflammatory Th17 lymphocytes.

Tregs are regulatory T cells (also referred to as “suppressor T cells”) that are generally immunosuppressive and can, for instance, help to prevent autoimmune diseases. Tregs can express several biomarkers, such as, for example, CD4 and forkhead box P3 (FOXP3). FOXP3 (also referred to as “scurfin”) is a protein that assists in regulation of regulatory pathways, including, for example, development of Tregs. Thus, the aforementioned CD4+FOXP3+ T regulatory cells are positive for (i.e., express) both CD4 and FOXP3.

MSC-sourced NO, in an autocrine manner, can increase IDO expression in MSC and significantly enhance their immunosuppressive properties. Additionally, MSC-derived PGE2 can attenuate the proliferation of activated T cells and prevent the conversion of naïve CD4+ T cells in effector Th1 and Th17 cells by suppressing IL-2 production in T lymphocytes. Moreover, MSC-sourced PGE2 can stimulate the generation of an immunoregulatory tolerogenic phenotype in DC and induce expansion of alternatively activated macrophages, contributing to the creation of an immunosuppressive microenvironment in inflamed tissues in which MSC are transplanted. Similar to PGE2, MSC-derived IL-10 and TGF-β can prevent the generation of inflammatory Th1 and Th17 cells by inhibiting the maturation of DC and by inducing the generation of alternatively activated (M2) phenotype in macrophages. Therefore, attenuated expression of co-stimulatory molecules (e.g., CD80 and CD86) and suppressed production of pro-Th1 and pro-Th17 cytokines (e.g., IL-12, IL-1β, IL-6, IL-23) can be observed in MSC-primed DC and macrophages.

In addition to T cells, DC, and macrophages, MSC are also able to efficiently inhibit proliferation and cytotoxicity of NK cells. MSC-derived TGF-β and NO can suppress the expansion of activated NK cells, while MSC-sourced IDO and PGE2 can generate the immunosuppressive and regulatory phenotype in NK cells. MSC-derived IL-10 can also down-regulate expression of pro-apoptotic and toxic molecules (e.g., perforins and granzymes) and inhibit the production of inflammatory and cytotoxic cytokines (e.g., TNF-α and IFN-γ) in NK cells, significantly reducing their cytotoxic potential.

Juxtacrine communication (e.g., direct cell-to-cell interaction between immune cells and MSC) may also be involved in MSC-dependent suppression of detrimental immunity. MSC can express pro-apoptotic molecules (e.g., programmed death-ligand (PDL)-1, PDL-2, Fas ligand (FasL)), which bind to PD and Fas receptors on the membranes of activated T and NK cells and can induce their apoptosis in a caspase-3-dependent manner.

Further, and specifically relating to MSC-based suppression in the therapy of oGVHD, intravenously injected BM-MSC may attenuate T cell-driven ocular inflammation, thereby alleviating DED in BM-MSC-treated oGVHD patients. This may result in, for instance, attenuated clinical symptoms (e.g., redness, ocular pain, dryness, scratchiness) and significantly decreased dry eye scores and/or ocular surface disease index scores. BM-MSC may prevent the activation of cytotoxic CD8+ T cells, resulting in a reduced number of CD28− expressing CD8+ T cells, which can be confirmed via, e.g., flow cytometry analysis of immune cells. Additionally, BM-MSC may alter the cytokine profile of activated CD8+ T cells. oGVHD patients that receive MSC and/or MSC-derived products (e.g., MSC-Exos) may exhibit a significantly reduced number of pro-inflammatory, IFN-γ, and IL-2-producing CD8+ T cells, and/or an increased presence of immunosuppressive, IL-10-producing CD8+ T cells. MSC-dependent immunoregulation may therefore result in these beneficial effects in oGVHD treatment. In certain instances, clinical improvements of DED-related symptoms may not occur in all BM-MSC-treated GVHD patients, which may occur if, for instance, BM-MSC are intravenously infused and are not injected directly into the eyes. In such situations, MSC-based immunomodulation would rely exclusively on the systemic effects of their secretome(s). This observation is in line with the potential therapeutic efficacy of subconjunctivally injected human BM-MSC in murine models of oGVHD. Subconjuctival transplantation of BM-MSC can completely attenuate detrimental immune response and significantly reduce oGVHD in MSC-treated animals. Massive intraocular infiltration of immune cells may be observed in saline-treated animals, but not in the eyes of BM-MSC-treated mice. Further, the total number of inflammatory CD3+ T cells and concentration of inflammatory TNF-α may be significantly reduced in the corneas of MSC-treated oGVHD animals. Additionally, subconjunctivally injected BM-MSC can suppress the expression of, e.g., the PAX6 gene in the corneas of oGHVD mice. Over-expression of the PAX6 gene can induce an altered morphology of corneal epithelial cells, increase corneal neovascularization, and promote intraocular infiltration of inflammatory immune cells, contributing to the progression of oGVHD. Accordingly, by potentially reducing expression of the PAX6 gene in the corneas of oGVHD mice, BM-MSC can alleviate intraocular inflammation, leading to the enhanced regeneration of injured corneal epithelial cells.

Transplantation of MSC in the Eyes of oGVHD Patients

Due to their potent regenerative and immunoregulatory properties, MSCs from all tissue sources can be used for the treatment of many incurable degenerative, autoimmune and inflammatory diseases, including, but not limited to, GVHD, oGVHD, other eye diseases, cancers, tumors, and other diseases and/or disorders. In experimental and clinical settings, MSCs can be injected either directly at the site of injury and inflammation (e.g., local transplantation) or systemically infused (e.g., intravenous, intra-arterial or intra-peritoneal injection). A majority of locally transplanted MSCs may become successfully engrafted at the site of injury where they can (i) secrete growth factors and provided trophic support to injured cells, (ii) produce immunoregulatory factors and suppress ongoing inflammation, and/or (iii) differentiate into parenchymal cells and repopulated damaged tissues. After intravenous injection, a majority of MSCs may engraft in the lungs and liver from where, in a paracrine and endocrine manner, through the activity of MSC-sourced immunomodulatory factors, the engrafted MSCs regulate detrimental immune response. Viability, phenotype, and/or function of systemically infused MSCs can be altered by cytokines to which they were exposed in systemic circulation and in the tissues of their engraftment. Therefore, significantly better therapeutic effects of MSCs may be observed after their direct transplantation in the injured and/or inflamed tissues. In line with these observations, the best therapeutic effects of MSCs in the treatment of oGVHD can be observed where these cells were topically administered, directly in the eyes of oGVHD patients.

In addition to MSCs, pluripotent stem cells (e.g., embryonic stem cells (ESC) and induced pluripotent stem cells (iPSCs)) can also be explored as a potentially valuable cell source for the repair and regeneration of injured epithelial cells in oGVHD patients. Under specific culture conditions, MSCs, ESCs, and iPSCs may have a similar potential for differentiation towards corneal epithelial cells. Importantly, compared to ESCs and iPSCs, MSCs may show a superior potential for immunoregulation and suppression of detrimental immune response in the eyes of oGVHD patients. However, ethical and safety issues related to the destruction of human embryos, undesired differentiation, and potential malignant transformation limit the clinical application of ESCs and iPSCs. Therefore, among all stem cells, only MSCs can be considered as novel therapeutic agents for the treatment of oGVHD.

It is important to recognize and prevent any possible safety issues which could limit the clinical use of MSC. Firstly, MSC are not “immune privileged” cells since they express MHC class II molecules. Accordingly, transplantation of allogeneic MSC may aggravate strong immune response elicited during oGVHD progression. Second, MSC are not exclusively immunosuppressive cells. MSC alter their phenotype and/or function under the influence of cytokines to which they are exposed. If MSC are transplanted at the ocular surface or in the vitreous body with a low level of TNF-α and IFN-γ, they can obtain a pro-inflammatory phenotype and secrete pro-inflammatory cytokines (e.g., TNF-α, IL-1β, IL-6, IL-12, IL-23), which could aggravate Th1 and Th17 cell-driven oGVHD. On the contrary, if MSC are engrafted in the eyes with ongoing inflammation (e.g., with the high levels of TNF-α and IFN-γ), they can acquire an immunosuppressive phenotype and produce, for instance, IDO, PGE2, IL-10, TGF-β, and/or other immunoregulatory factors that efficiently attenuate detrimental immune response. In line with these findings, there is an objective concern that MSC transplanted in the eyes with a low concentration of TNF-α and IFN-γ will obtain a pro-inflammatory phenotype and will aggravate oGVHD. Additionally, TGF-β and bone morphogenetic proteins (BMPs), released by macrophages and parenchymal cells in inflamed eyes, may induce unwanted chondrogenic and osteogenic differentiation of transplanted MSC. Although measurement of inflammatory cytokines and growth factors in the eyes of oGVHD patients prior to MSC injection will minimize the risk for MSC-dependent aggravation of oGVHD, it should be noted that intraocular levels of TNF-α, IFN-γ, and TGF-β can dynamically change during the progression of oGVHD and, therefore, concentrations of these cytokines should be continuously monitored in all MSC-treated oGVHD patients.

Therapeutic Potential of MSC-Exos in the Treatment of oGVHD

The majority of MSC-sourced immunoregulatory and growth factors that suppress detrimental immune response in the eyes and support regeneration of injured corneas, conjuctivas, Meibomian, and lacrimal glands are contained within MSC-sourced exosomes (MSC-Exos) that can be, for instance, 50-150 nm large. As cell free products, MSC-Exos address all safety concerns related to the transplantation of MSC. Furthermore, the lipid bilayer of the MSC-Exos' membrane enables ease of penetration of MSC-Exos through the corneal epithelium and across the blood-retina barrier. Accordingly, topical administration of MSC-Exos has been considered as an alternative therapeutic approach to MSC-based therapy in the treatment of oGVHD.

MSC-Exos from all tissue sources are enriched with MSC-sourced microRNA (miRNAs), which bind to the RNA-induced silencing complex and inhibit gene expression in target cells. For instance, MSC-Exo-sourced miR-10a-5p and miR-10b-5p can prevent the apoptosis of injured epithelial cells, miR-191-5p can facilitate cell viability of limbal stem cells, while MSC-derived miR146a can suppress detrimental immune response by down-regulating expression of IFN-γ in Th1 lymphocytes. Labial gland MSC-Exo-sourced miR-125b can affect antibody secretion in plasma cells of patients suffering from DED secondary to Sjogren's Syndrome by modulating expression of the PR domain zinc finger protein 1 (PRDM1) gene. Accordingly, MSC-Exos may significantly reduce the percentage of activated, antibody producing, CD19+CD20−CD27+CD38+ plasma cells in peripheral blood mononuclear cells of these patients and attenuated antibody-dependent injury of lacrimal glands.

AF-MSC-Exos are enriched in neurotrophins (e.g., NGF, brain derived growth factor (BDNF)) which provide trophic support to injured neurons and promote axonal regeneration, crucially contributing to the retinal regeneration in the eyes of oGVHD patients.

AT-MSC-Exos also contain cytokines and growth factors that regulate lymphocyte activation (e.g., IL-10, IL-1Ra, TGF-β, GRO, soluble TNF-α receptors (sTNFRs)), and promote repair and/or regeneration of injured tissues (e.g., MMP-2 and 9). In line with these findings, MSC-Exos-mediated immunosuppression may be mainly responsible for attenuation of DED in BAC+MSC-Exo-treated mice. Specifically, AT-MSC-Exos may improve viability of injured epithelial cells by suppressing caspase-1-driven apoptosis. Additionally, AT-MSC-Exos can inhibit activation of NLR family pyrin domain containing 3 (NLRP3) inflammasome and suppress the expression of IL-1β and IL-18 in lacrimal gland-infiltrated macrophages, which can significantly reduce ocular inflammation and attenuated DED in experimental mice.

In line with these findings, topical administration of human AF-MSC-Exo-sourced eye drops in clinical settings may efficiently attenuate pain, dryness, grittiness, scratchiness, soreness, irritation, burning, watering, and/or eye fatigue in DED patients. AF-MSC-Exo-sourced eye drops can contain, for instance, IL-1Ra, sTNFRI, STNFRII, GRO-γ, fatty acid-binding protein 1 (FABP1), and platelet factor 4 (PF4), which suppress IL-1β and TNF-α-driven inflammation, prevent the generation of inflammatory Th1 and Th17 cells, support tear stability, and reduce ocular surface epithelial damage in patients suffering from inflammatory eye diseases. AF-MSC-Exo-sourced eye drops may also promote regeneration of injured meibomian glands and restore meibomian function in patients suffering from, e.g., MGD. Before topical application of AF-MSC-Exo-sourced eye drops, meibomian ducts of MGD patients may be dilated while meibomian glands may be enlarged and tortuous with abnormal structure. The morphology of meibomian glands can accordingly significantly improve after a specific duration (e.g., 21 days) of AF-MSC-Exo-based therapy, showing the hypoilluminescent grape-like clusters. Similarly, hyperilluminescent ducts and underlying tarsus can indicate beneficial effects of AF-MSC-Exos in restoration of meibomian gland and ducts morphology. For instance, significantly improved tear film breakup time may be observed a specific duration (e.g., 21 days) after topical administration of AF-MSC-Exo-sourced eye drops, confirming restoration of meibomian gland function. Similarly, significantly improved visual acuity, relieved ocular pain, and complete healing of corneal epithelial defects may be noticed in AF-MSC-Exo-treated patients that suffer from Sjogren's syndrome. In addition to these findings, AF-MSC-Exo-sourced eye drops can improve the viability of injured corneal epithelial cells and alleviate the symptoms elicited by corneal injury. A specific course of AF-MSC-Exo-based therapy (e.g., four weeks) can remarkably improve visual acuity and significantly decrease ocular pain in patients suffering from epithelial basement membrane dystrophy with recurrent corneal erosion syndrome (RCES). Such improvement may include, for instance, no recurrence of RCES symptoms in AF-MSC-Exo-treated patients after four months, suggesting beneficial effects of AF-MSC-Exos in the repair and regeneration of injured corneal epithelial cells. Importantly, AF-MSC-Exo-sourced eye drops ae believed to be generally well tolerated in clinical settings, without any side effects.

Only one registered clinical trial (NCT04213248) which is going to investigate therapeutic potential of UC-MSC-Exos in the treatment of oGVHD is currently recruiting patients. Patients will receive artificial tears for 2 weeks to normalize the baseline, and afterwards, UC-MSC-Exo eye drops (10 μg/drop; four times a day) will be administered for 14 days. Changes in ocular surface disease index, conjunctiva redness scores, tear secretion, tear break time, ocular surface staining, best corrected visual acuity, and tear meniscus height will be determined during the follow-up of 12 weeks. It is expected that the first results of this study will be published in next two years.

Therapeutic Potential of MSC-Exos May Depend on Tissue Origin of their Parental Cells

It should be noted that the content, and therefore, therapeutic potential of MSC-Exos may depend on the tissue origin of their parental cells. For instance, BM-MSC-Exos may be enriched with immunoregulatory factors that induce the generation of an immunosuppressive phenotype in macrophages (e.g., TGF-β, IL-10), protect from oxidative stress-induced injury (e.g., miR-214), attenuate TNF-α and IL-1β-driven inflammation (e.g., sTNFRI, sTNFRII, IL-1Ra), promote expansion of Tregs, and prevent IL-23-dependent generation of Th17 cells (e.g., IDO, Kynurenine, TGF-β). UC-MSC-Exos may contain the enzymes manganese superoxide dismutase and glutathione peroxidase 1, which have anti-apoptotic and anti-oxidation abilities and are capable of preventing oxidative stress-induced injury of neural cells in the eyes of oGVHD patients. Additionally, UC-MSC-Exos may reduce nerve inflammation since they are enriched in proteins which block the degradation and proliferation of the NFκB inhibitor IκBα. UC-MSC-Exos can also contain miR-21, miR-23a, miR-125b, and/or miR-145, which inhibit fibrosis by affecting the factor-β2/SMAD2 pathway, and miR-135b-5p and/or miR-499a-3p, which regulate angiogenesis. AF-MSC-Exos may contain proteins that modulate neurodevelopment and lymphocyte activation (e.g., A disintegrin and a metalloprotease (ADAM)-9, ADAM-10), repair and regeneration of injured tissues (e.g., MMP-2 and 9), and are enriched in proteins that regulate oxidative stress e.g., (peroxiredoxin-1,-2,-4,-6). AF-MSC-Exos can also contain neurotrophins, which provide trophic support to injured neurons in the eyes of oGVHD patients. Therefore, AT-MSC-Exos may have similar therapeutic potential for oGHVD treatment as other tissue source MSC-Exos. AT-MSC-Exos may also be enriched with immunoregulatory proteins, such as, for example, IDO, TGF-β, IL-10, IL-1Ra, and PGE2, which suppress Th1 and Th17 cell-driven inflammation and NK cells-dependent injury of epithelial cells in oGVHD patients. The main advantage of AT-MSC-Exos is their high availability, since they are easily derived from oGVHD patients' AT. Accordingly, AT-MSCs may be an alternative when MSC-Exos from other sources are difficult to extract or are not suitable for therapy.

Although MSC-Exos from all tissue sources represent potentially effective therapeutic agents in regenerative ophthalmology, the exact therapeutic dose of MSC-Exos for oGVHD treatment is still unknown. Therefore, the optimal dose, frequency, and treatment schedule must be determined before MSC-Exos can be offered as a new remedy for oGVHD treatment. Additionally, the exact growth and immunoregulatory factor(s) which is/are mainly responsible for the beneficial effects of MSC-Exos in the therapy of oGVHD should be defined. Afterwards, MSCs could be genetically engineered to over-express these factors, which will be contained at high concentrations in MSC-Exos. Administration of MSC-Exos enriched with the most effective bioactive factor(s) will enhance the therapeutic potential and efficacy of MSC-Exos in the treatment of oGVHD.

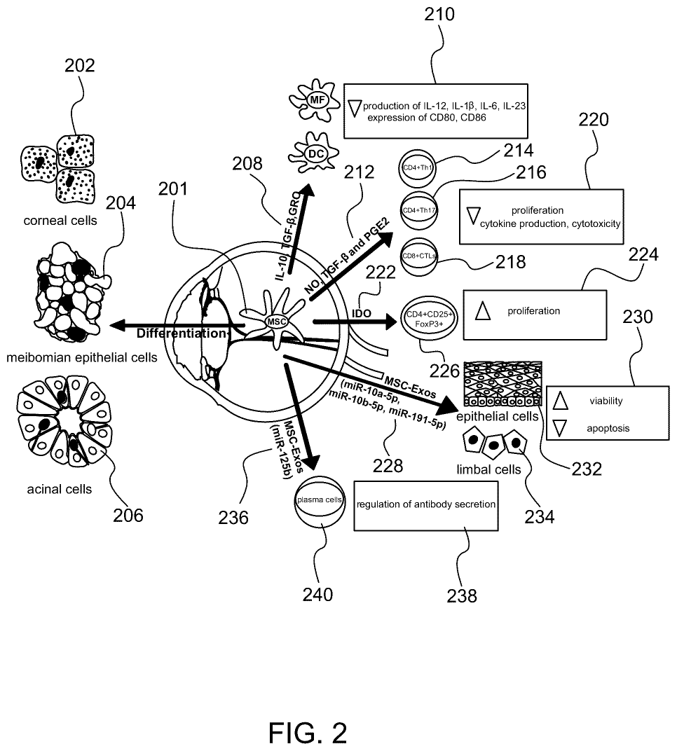

Thus, due to their capacity for differentiation in corneal epithelial cells and because of their immunosuppressive properties, MSC can enhance repair and regeneration of the epithelial barrier at the ocular surface, suppress eye inflammation, and restore meibomian and lacrimal glands' function, as shown in FIG. 2. MSC 201 differentiate into corneal cells 202, meibomian epithelial cells 204, and acinal cells 206 of lacrimal glands. MSC-derived IL-10, TGF-β, GRO (collectively 208) cause effects 210, including attenuated expression of co-stimulatory molecules (CD80 and CD86) and suppressed production of IL-12, IL-1β, IL-6, IL-23 in macrophages and DCs. MSC-derived NO, TGF-β, and PGE2 (collectively 212) suppress proliferation and expansion of inflammatory CD4+ Th1 cells 214 and Th17 cells 216 and cytotoxic CD8+ T cells 218. Further, effects 220 occur, including decreased proliferation, cytokine production, and cytotoxicity. MSC-derived IDO 222 induces expansion 224 of immunosuppressive Tregs 226. MSC-Exo-sourced miR-10a-5p, miR-10b-5p and miR-191-5p (collectively 228) cause effects 230, such as prevention of apoptosis and facilitation of viability of injured epithelial cells 232 and limbal stem cells 234. MSC-Exo-sourced microRNA-125b 236 regulates antibody secretion 238 in plasma cells 240 in the eyes of oGVHD patients.

Table 3 below shows the therapeutic potential of different types of MSCs and MSC-Exos in oGVHD treatment. The relevant animal models and/or patients are also listed, along with a specific, non-limiting route of injection, the relevant mechanism(s) of action, and non-limiting beneficial effects.

TABLE 3
Therapeutic potential of MSCs and MSC-Exos in oGVHD treatment

| Animal | Type of | | | |
|---|---|---|---|---|
| Models/ | MSCs/MSC- | Route of | Mechanisms | Beneficial |
| Patients | Exos | Injection | of Action | Effects |
| rabbit model of | BM-MSCs | intraocular | differentiation in | regeneration of |
| corneal injury | | | corneal epithelial | corneal epithelium |
| | | | cells | |
| mice model of LSCD | AT-MSCs | intraocular | repopulation of | regeneration of |
| | | | limbal stem cells | corneal epithelium |
| patients with LSCD | BM-MSCs | intraocular | repopulation of | regeneration of |
| | | | limbal stem cells | corneal epithelium |
| rat model of DED | BM-MSCs | intraocular | suppression of | regeneration of |
| | | | eye inflammation | meibomian glands |
| mice model of | LG-MSCs | intraocular | suppressed apoptosis | restoration of lacrimal |
| aqueous-deficient | | | of acinar cells | glands' structure |
| DED | | | | |
| patients with GVHD | BM-MSCs | intravenous | MSC-dependent suppression | reduced injury of |
| associated with DED | | | of CD8 + CTLs | epithelial cells |
| mice model of oGVHD | BM-MSCs | subconjunctival | MSC-dependent suppressed | attenuated eye inflammation; |
| | | | expression of PAX6 gene in | enhanced regeneration of |
| | | | corneas; inhibition of | injured corneal epithelial |
| | | | eye-infiltrated CD3 + | cells |
| | | | T cells; down-regulated | |
| | | | production of TNF-α | |
| | | | in the eyes | |
| mice model of primary | Labial gland- | intravenous | miR-125b-dependnet | alleviated antibody- |
| Sjogren syndrome | MSC-Exos | | modulation of PRDM1 | dependent injury of |
| | | | expression in | epithelial cells |
| | | | plasma cells | |
| mice model of DED | AT-MSC-Exos | intraocular | suppression of NLRP3 in | attenuated eye inflammation; |
| | | | macrophages | restoration of corneal |
| | | | | epithelium |
| patients | AF-MSC-Exos | intraocular | inhibition of eye- | reduced DED-related |
| with DED | | | infiltrated Th1, Th17 cells | symptoms |

MSC-Exos, and/or any compound and/or formulation containing MSC-Exos (e.g., d-MAPPS pharmaceutical compositions including, for instance, d-MAPPS solutions), which contain all MSC-sourced growth factors and immunoregulatory proteins, can easily bypass all biological barriers in the eyes due to their nano-size dimension and lipid envelope. Accordingly, such MSC-Exos can deliver their cargo directly in corneal epithelial cells and eye-infiltrated leukocytes. As a cell-free agent, MSC-Exos address all relevant safety issues related to the transplantation of their parental cells, including the risk of unwanted differentiation and aggravation of intraocular inflammation.

Accordingly, in at least one embodiment of the disclosure, d-MAPPS pharmaceutical compositions (e.g., d-MAPPS solutions) containing one or more MSCs and/or one or more MSC-Exos are disclosed. Such d-MAPPS pharmaceutical compositions may include any one or more types of MSCs described herein, one or more types of MSC-Exos described herein, and/or one or more MSC-sourced growth factors and/or immunoregulatory proteins described herein.

Compounds and/or Formulations Containing De-Cellularized Human Amniotic Fluid

In at least one embodiment of the disclosure, d-MAPPS pharmaceutical compositions (e.g., d-MAPPS solutions) include sterile de-cellularized human amniotic fluid (D-HAF), either in fluid form or solid form, for example, lyophilized powder, alone or in combination with appropriate excipients. Other active agents can be included. D-HAF contains over 300 human growth factors. D-HAF is devoid of amniotic stem cells and elements of micronized membrane or chorion particles.

Amniotic fluid (“AF”) contains nutrients and growth factors that facilitate fetal growth, provides mechanical cushioning and antimicrobial effectors that protect the fetus, and allows assessment of fetal maturity and disease. AF typically contains mixtures of growth factors, pro-inflammatory cytokines and anti-inflammatory cytokines, as well as a variety of macromolecules including carbohydrates, proteins and peptides such as enzymes and hormones, lipids, lactate, pyruvate, and electrolytes.

In some embodiments, the raw fluid directly collected from the source is not heat-treated, chemical-treated, or fractionated to produce the disclosed formulations. In some embodiments, the formulation retains more than 50%, more than 60%, more than 70%, more than 80%, or preferably more than 90%, of the total amniotic factors present in the raw fluid. In some embodiments, the formulations are not diluted with any additional solution for storage. In some embodiments, the formulations are diluted prior to application to the eyes. In some embodiments, the formulations are not concentrated relative to the raw fluid.

In some embodiments, the d-MAPPS composition (e.g., one or more d-MAPPS solutions) includes a diluted sterile de-cellularized human amniotic fluid (D-HAF), which preferably has not been heat treated, typically administered using a standard eye dropper apparatus. D-HAF contains over 300 human growth factors. D-HAF is devoid of cells, including amniotic stem cells, and elements of micronized membrane or chorion particles. The purified fluid is sterilized without the use of harsh terminal irradiation, e-beam or Ethylene Oxide (EO). In at least a further embodiment, the process includes separating the cells from the AF using centrifugation and utilizing a series of filtration devices to remove all remaining cells and bioburden. Each lot is tested for bioburden and is certified sterile to contain <1 harmful organisms.

Generally, methods of preparing sterile de-cellularized amniotic fluids involve a series of centrifugation and filtration steps. Preferred methods of preparing sterile de-cellularized amniotic fluid are described in detail in U.S. application Ser. No. 15/053,497.

### Method of Preparation

In some embodiments, D-HAF is prepared from sterile human amniotic fluid obtained from a woman, removing cells, large particles and other undissolvables are removed, preferably by high speed centrifugation to obtain clarified amniotic fluid, the clarified amniotic fluid filtered through filters having a pore size of about 5 μm to about 10 μm to obtain a micron filtrate, filtering the micron filtrate through filters with a pore size of about 1.0 μm to obtain a second filtrate, filtering the filtrate through submicron filters with the pore size of 0.45 μm or/and 0.2 μm to obtain the sterilely filtered amniotic fluid.

In some embodiments, a collection procedure is performed in a sterile operating room environment during an elective C-section. Typically, the woman is undergoing a pre-Caesarian surgical procedure. The steps of obtaining the sterile human amniotic fluid includes the steps of turning on a ultrasound device to provide guidance for the process of obtaining human fluid from the woman, inserting a blunt tip needle into the amniotic sac of the woman, attaching the blunt tip needle to a three-way stopcock, connecting a Luer lock syringe to the three-way stopcock, connecting a first end of a length of sterile tubing with the three-way stopcock, and collecting sterilely the amniotic fluid through the blunt tip needle and sterile tubing into a collection container.

In this embodiment, the sterile collection container includes a pump with a suction device. The suction device is a low suction device or a spring loaded low suction device. The suction device is fluidly connected to an internal balloon. This embodiment further includes manually pumping up the internal balloon in the sterile collection container using the low suction device to allow a low-level suction and collection of the amniotic fluid.

In some embodiments, the step of removing cells, large particles and other undissolvables from the human amniotic fluid includes a first step of centrifuging or depth filtering the human amniotic fluid. In some embodiments, the human amniotic fluid is centrifuged at about 5,000 rpm to about 10,000 rpm for about 30 minutes to about 60 minutes. In this embodiment, filters of about 5 μm to about 10 μm are used for the first step. These can be cellulose ester filters, glass fiber filters, nylon capsule filters or nylon cartridge filters. The filters with the pore size of 1.0 μm are capsule filters or cartridge filters. The filters with the pore size of 1.0 μm are poly ether sulfone, poly vinylidene fluoride or cellulose acetate membrane filters. The filters with the pore size of 0.45 μm or 0.2 μm are capsule filters or cartridge filters. The filters with the pore size of 0.45 μm or 0.2 μm are poly ether sulfone membrane filters, poly vinylidene fluoride or cellulose acetate membrane filters.

The sterilely filtered human amniotic fluid contains growth factors including human growth hormone, transforming growth factor beta 1, vascular endothelial growth factor, epidermal growth factor, transforming growth factor beta 3, and growth differentiation factor 11 or combinations thereof.

In some embodiments, the process of obtaining the sterile amniotic fluid further includes the step of lyophilizing the sterile amniotic fluid to obtain a lyophilisate. The lyophilisate can be further sterilized by e-beam irradiation or gamma ray irradiation to reinforce the sterility.

Tools to obtain sterilely filtered human amniotic fluid from a woman, include a three-way stopcock, a sterile blunt tip needle aseptically attached to the three-way stopcock, a Luer lock syringe aseptically connected to the three-way stopcock, a sterile tubing aseptically connected to the three-way stopcock, a collection container or a collection container including a pump with suction device connected with the sterile tubing, a set of filters having the pore size of about 5 μm to about 10 μm, a set of capsule or cartridge filters having the pore size of about 1 μm, a set of capsule or cartridge filters having the pore size of about 0.45 μm or 0.2 μm, a set of sterile syringes or vials to store the sterile filtered amniotic fluid and operating instructions on using the kit to obtain sterilely filtered human amniotic fluid. The filters having the pore size of from about 5 μm to about 10 μm and the capsule or cartridge filters are made from cellulose ester, glass fiber or nylon. The sterile collection container may include a pump with a suction device. The suction device may be a low suction device or spring loaded low suction device. In another aspect the suction device may be fluidly connected to an internal balloon. Further to this aspect the method includes manually pumping up the internal balloon in the sterile collection container using the low suction device to allow a low-level suction and collection of the amniotic fluid. In yet another aspect the sterile collection container may include an inlet. Further to this particular aspect the method includes connecting a second end of the tubing to the inlet of the sterile collection container. The sterile collection container may include a vent having a cap.

In some embodiments, utilizing the incision site immediately prior to performing the C-section and with ultrasound guidance to protect the fetus and mother provides a minimal or no risk environment for collection.

Collection is achieved via a low level suction established within a collection container and/or via gravity. Typically, after high speed centrifugation, filtration with 5 to 10 μm filters (low protein binding filter) is used to complete the removal of cells and large particles. Submicron filtration is then conducted with 1 μm and 0.45 μm or/and 0.2 μm filters (low protein binding filter), two in a series connection, to remove gross contaminates. Under this condition, soluble growth factors will pass through this filter to achieve a semi-sterile condition, very low bioburden counts. If under a strict aseptic operation condition, a 10⁻³ sterility assurance level is achieved. A 10⁻⁶ sterility assurance level can be achieved by submicron filtration with a 0.22 μm filter (low protein binding filter) at the end and sterile packaging to achieve a sterile product. One can monitor the filtrate after each filtration step to determine which components are removed and then to determine which process to use to achieve the desirable product.

One may use membrane filters including or made of hydrophilic polyethersulphone (PES) to filter protein solutions. Filter disks for small volumes and different sizes of cartridges for larger volumes such as 1 liter and more are used. Hydrophobic membranes like PTFE which are designed for liquids devoid of proteins should not be used. Start with centrifugation at 5000 to 8000 rpm for at least 30 minutes. Next, the supernatant is filtered with a prefilter to remove residual protein aggregates and precipitates in suspension (AP20 can be used). If one directly uses a 0.6/0.2 μm filter, after prefiltration, one may experience slow filtration rates and the flow may stop too quickly. It may be desirable to make intermediate filtration steps using 1.2 μm and 0.8 μm membranes. Typically, a final filtration through 0.2 μm is necessary to get the best sterility assurance level and produce a sterile amniotic fluid for injections. The final filtrate can be stored in frozen condition at about −20° C. to about −80° C. for long term storage. In addition, the sterilely filtered amniotic fluid may be distributed in vials equipped with special rubber stoppers for sterile lyophilization.

The sterile amniotic fluid can be lyophilized to yield a lyophilisate. The sterilely filtered amniotic fluid may be distributed in vials equipped with special rubber stoppers for sterile lyophilisation. The lyophilisation is carried out in a sterile environment. The rubber stoppers on the vials are then automatically pushed down in the freeze dryer to definitively close them. Finally an aluminum cap is sealed on each vial to protect its sterile content. In such a lyophilized state, the amniotic fluid may be stored at +4° C. or room temperature for at least one year without decrease of its biological activity. The lyophilisate can be irradiated by e-beam irradiation or gamma ray irradiation to insure the sterility. For its medical use, the sterile amniotic fluid may be reconstituted by adding the initial volume of sterile water to the powder in order to restore a transparent and homogeneous physiological liquid.

### Sources of Amniotic Fluid

In some embodiments, d-MAPPS compositions (e.g., d-MAPPS solutions) that include amniotic fluid are prepared, at least in part, from sterile human amniotic fluid obtained from a pregnant woman.

Suitable sources, e.g. of human AF, include AF that is obtained from patients who are undergoing amniocentesis, patients who are undergoing a Caesarean section delivery, and patients undergoing normal delivery using a specially designed receptacle to collect the fluid after rupture of membranes.

D-HAF, and/or d-MAPPS compositions and/or d-MAPPS solutions containing D-HAF, can be stored for long periods of time, allowing for a broad range of application methods, including distribution and storage as aerosols, solutions, powders, etc. In some embodiments, d-MAPPS compositions and/or d-MAPPS solutions containing D-HAF is refrigerated at about 1° C. to about 10° C. for long-term storage. In a further embodiment, d-MAPPS compositions and/or d-MAPPS solutions containing D-HAF is refrigerated at 4° C. for up to 12 months and more. Preferably, the long-term storage does not reduce the quantity of the total soluble proteins or factors present in the D-HAF. For some embodiments, the total soluble proteins retained after long-term storage in refrigerated conditions is about 10%, 20%, 30%, 40%, 50%, 60%, 70%, 80%, or 90% of the fresh D-HAF.

D-HAF, and/or d-MAPPS compositions and/or d-MAPPS solutions containing D-HAF, can be supplied as a clear one-part solution in a suitable container for storage at 4° C., or for storage at −20° C., or at −80° C. For example, liquid formulations in prefilled aliquots can be suitable for storage at 1-5° C., or for storage at −20° C., or at −80° C. The liquid formulation can be suitable for topical application in a nebulizer or a spray. In other embodiments, the fluid can be supplied as a kit that can be stored at 4° C., at −20° C., or at −80° C. until needed.

In some embodiments, D-HAF, and/or d-MAPPS compositions and/or d-MAPPS solutions containing D-HAF, use a final filtration through 0.2 μm to produce a sterile amniotic fluid without any irradiation. In some embodiments, D-HAF, and/or d-MAPPS compositions and/or d-MAPPS solutions containing D-HAF, have a 10⁻⁶ sterility assurance level without irradiation. In other embodiments, lyophilisate derived from amniotic fluid through lyophilisation may be irradiated by e-beam irradiation or gamma ray irradiation to add another guarantee for the final sterility of the powder.

### Growth Factors, Cytokines, and Other Molecules

Growth factors and their receptors control a wide range of biological functions, regulating cellular proliferation, survival, migration and differentiation. Growth factors found in AF play a critical role in fetal growth and development.

A non-limiting list of growth factors that have been identified in AF includes factors such as epidermal growth factor (EGF), insulin-like growth factor I (IGF-I), vascular endothelial growth factor A (VEGF-A), tumor necrosis factor A (TNF-α), hepatocyte growth factor (HGF), fibroblast growth factor 7 (FGF7), matrix metallopeptidase (MMP-9), granulocyte-colony stimulating factor (GCSF), matrix metalloproteinase-7 (MMP-7), matrix metalloproteinase-13 (MMP-13), transforming growth factor alpha (TGF-α), transforming growth factor beta (TGF-β), fibroblast growth factor 4 (FGF-4), endocrine gland-derived vascular endothelial growth factor (EG-VEGF), interleukin 8 (IL-8), fibroblast growth factor 21 (FGF-21), angiopoietin-2 (ANG-2), Glial cell-derived neurotrophic factor (GDNF), fibroblast growth factor 19 (FGF-19), TIMP metallopeptidase inhibitor 2 (TIMP-2), angiopoietin-1 (ANG-1), transforming growth factor beta 1 (TGFβ1), macrophage colony-stimulating factor (M-CSF), angiotensinogen, platelet derived growth factor-AA (PDGF-AA), and stem cell factor (SCF).

Epidermal growth factor (EGF) is a small polypeptide hormone with mitogenic properties in vivo and in vitro. EGF elicits biologic responses by binding to a cell surface receptor which is a transmembrane glycoprotein containing a cytoplasmic protein tyrosine kinase. EGF responses are mediated by ligand binding and activation of this intrinsic protein kinase. The receptor can be phosphorylated by other protein kinases, and this may regulate receptor function. Stimulation of the receptor tyrosine kinase activity by ligand binding must regulate the activity of an as yet undefined molecule(s) responsible for transmitting a mitogenic signal to the nucleus (Todderud G., et al., Biofactors. 1989, 2(1): 11-5).

Vascular endothelial growth factor (VEGF), also known as vascular permeability factor (VPF), was originally described as an endothelial cell-specific mitogen. VEGF is produced by many cell types including tumor cells, macrophages, platelets, keratinocytes, and renal mesangial cells. The activities of VEGF are not limited to the vascular system; VEGF plays a role in normal physiological functions such as bone formation, hematopoiesis, wound healing, and development (Duffy A. M., et al., In: Madame Curie Bioscience Database [Internet]. Austin (TX): Landes Bioscience (2000)).

TGF-α has a structure similar to EGF and binds to the same receptor. The amnion cells of the umbilical cord express EGF, TGF-α, and the functional EGF/TGF-α receptor, suggesting the possibility of a regulating role of the amnion in fetal growth and development. EGF and TGF-α have also been shown to stimulate the production of surfactant components.

TGFβ1 is believed to induce terminal differentiation of intestinal epithelial cells and to accelerate the rate of healing of intestinal wounds by stimulating cell migration. TGFβ1 may also stimulate IgA production. VEGF-A is a signal protein that stimulates vasculogenesis and angiogenesis (Hoeben Am, et al., Pharmacol Rev. 2004, 56:549-580).

Transforming growth factor-beta (TGF-β) is a multifunctional peptide that controls proliferation, differentiation, and other functions in many cell types. Many cells synthesize TGF-β and essentially all of them have specific receptors for this peptide. TGF-β regulates the actions of many other peptide growth factors and determines a positive or negative direction of their effects (Sporn M. B., et al., Science 1986, 233(4763) 532-534).

Hepatocyte growth factor (HGF), the ligand for the receptor tyrosine kinase encoded by the c-Met proto-oncogene, is a multidomain protein structurally related to the pro-enzyme plasminogen and with major roles in development, tissue regeneration and cancer. A recent study showed its immunomodulation potential of amniotic fluid stem cells (Maraldi T., et al., Stem Cells Transl. Med., 4(6):539-47 (2015)).

Fibroblast growth factors (FGFs) that signal through FGF receptors (FGFRs) regulate a broad spectrum of biological functions, including cellular proliferation, survival, migration, and differentiation. The FGF signal pathways are the RAS/MAP kinase pathway, PI3 kinase/AKT pathway, and PLCγ pathway, among which the RAS/MAP kinase pathway is known to be predominant. Several studies have recently implicated the in vitro biological functions of FGFs for tissue regeneration. Many current applications of FGF are in regeneration of tissues, including skin, blood vessel, muscle, adipose, tendon/ligament, cartilage, bone, tooth, and nerve tissues (Yun Y. R., et al., J. Tissue Eng. 2010: 1(1)).

Matrix metalloproteinases (MMPs), also called matrixins, function in the extracellular environment of cells and degrade both matrix and non-matrix proteins. They play central roles in morphogenesis, wound healing, tissue repair and remodeling in response to injury, e.g., after myocardial infarction, and in progression of diseases such as atheroma, arthritis, cancer and chronic tissue ulcers. They are multi-domain proteins and their activities are regulated by tissue inhibitors of metalloproteinases (TIMPs) (Nagase H., et al., Cardiovascular Research, European Society of Cardiology, 562-573 (2006)).

Amniotic fluid also contains many pro- and anti-inflammatory cytokines. Pro- and anti-inflammatory cytokines play important immunoregulatory roles. Inflammation is characterized by interplay between pro- and anti-inflammatory cytokines. Cytokines are commonly classified in one or the other category: interleukin-1 (IL-1), tumor necrosis factor (TNF), gamma-interferon (IFN-γ), IL-12, IL-18, and granulocyte-macrophage colony stimulating factor are well characterized as pro-inflammatory cytokines, whereas IL4, IL-10, IL-13, IFN-α and TGF-β are recognized as anti-inflammatory cytokines.

Exemplary pro-inflammatory cytokines include Eotaxin-2 (CCL24), interleukin 6 (IL-6), pulmonary and activation-regulated chemokine PARC or chemokine (C-C motif) ligand 18 (CCL18), total GRO which consisted of three subunits GROα/CXCL1, GROβ/CXCL2, and GROγ/CXCL3, expression of the neutrophil-activating CXC chemokine (ENA-78/CXCL-5), chemokine (C-C motif) ligand 21 (CCL21or 6Ckine), macrophage inflammatory protein 3 alpha (MIP-3a or CCL20), monokine induced by gamma (MIG or CXCL-9), MIP-1α, chemokine (C-C motif) ligand 5 (CCL-5), also known as RANTES (regulated on activation, normal T cell expressed and secreted), Interleukin-1 alpha (IL-1α), macrophage inflammatory protein-1β (MIP-1β or CCL4), tumor necrosis factor (TNF-α), and monocyte chemotactic protein 2 (MCP-2 or CCL8).

Exemplary anti-inflammatory cytokines include interleukin 8 (IL-8), interleukin 13 (IL-13), interleukin 27 (IL-27), cytotoxic T-lymphocyte-associated protein 4 (CTLA-4), vascular endothelial growth factor D (VEGF-D), interleukin-1 receptor antagonist (IL-IRa), transforming growth factor beta 1 (TGFβ1), interleukin 5 (IL-5), and interleukin 21 (IL-21).

Additional Therapeutic, Prophylactic, and/or Diagnostic Agents

In some embodiments, d-MAPPS compositions (e.g., d-MAPPS solutions that include MSC, MSC-Exos, and/or one or more MSC-sourced growth factors and/or immunoregulatory proteins) are used in combination with one or more additional therapeutic, diagnostic, and/or prophylactic agents to alleviate pain (e.g., pain associated with one or more diseases, including, for instance, eye diseases), facilitate healing, and/or to reduce or inhibit scarring. In at least an additional embodiment, the d-MAPPS compositions (e.g., d-MAPPS solutions that include MSC, MSC-Exos, and/or one or more MSC-sourced growth factors and/or immunoregulatory proteins) comprise one or more additional compounds to prevent or treat one or more eye diseases (e.g., oGVHD), and/or to relieve symptoms such as inflammation. Non-limiting examples include antimicrobial agents, analgesics, local anesthetics, anti-inflammatory agents, antioxidants, immunosuppressants, anti-allergenic agents, enzyme cofactors, essential nutrients, and growth factors.

In some cases, one or more additional active agents may be dispersed in, or otherwise associated with particles in, d-MAPPS compositions (e.g., d-MAPPS solutions that include MSC, MSC-Exos, and/or one or more MSC-sourced growth factors and/or immunoregulatory proteins). In certain embodiments, one or more additional active agents may also be dissolved or suspended in the pharmaceutically acceptable carrier.

In at least one embodiment, the active agents include, for instance, small molecules, biomolecule, peptides, sugar, glycoproteins, polysaccharides, lipids, nucleic acids, and/or combinations thereof. Suitable small molecule active agents include, but are not limited to, organic and organometallic compounds. In at least one instance, the aforementioned small molecule active agent has a molecular weight of less than about 2000 g/mol, more preferably less than about 1500 g/mol, and most preferably less than about 1200 g/mol. The small molecule active agent can be a hydrophilic, hydrophobic, or amphiphilic compound. In at least one example, one or more additional agents may be dispersed, dissolved, and/or suspended in one or more d-MAPPS compositions (e.g., d-MAPPS solutions that include MSC, MSC-Exos, and/or one or more MSC-sourced growth factors and/or immunoregulatory proteins).

In some cases, the active agent is a diagnostic agent imaging or otherwise assessing the eye. Exemplary diagnostic agents include paramagnetic molecules, fluorescent compounds, magnetic molecules, and radionuclides, x-ray imaging agents, and contrast media.

When used for the treatment of ocular diseases, d-MAPPS compositions (e.g., d-MAPPS solutions that include MSC, MSC-Exos, and/or one or more MSC-sourced growth factors and/or immunoregulatory proteins) may contain one or more ophthalmic drugs to treat, prevent or diagnose a disease or disorder of the eye. Non-limiting examples of ophthalmic drugs include anti-glaucoma agents, anti-angiogenesis agents, anti-infective agents, anti-inflammatory agents, an analgesic, a local anesthetic, growth factors, immunosuppressant agents, anti-allergic agents, an anti-oxidant, a cytokine, and combinations thereof.

Volume of administration of one or more d-MAPPS compositions (e.g., d-MAPPS solutions that include MSC, MSC-Exos, and/or one or more MSC-sourced growth factors and/or immunoregulatory proteins) is tissue-specific and dependent on the disease, disorder, and/or condition to be treated. Dosages can be readily determined by those of skill in the art. See, e.g., Ansel et al., Pharmaceutical Dosage Forms and Drug Delivery Systems (6th ed.), Williams and Wilkins (1995). Additionally, one or more d-MAPPS compositions (e.g., d-MAPPS solutions that include MSC, MSC-Exos, and/or one or more MSC-sourced growth factors and/or immunoregulatory proteins) may be administered in conjunction with other types of cells, e.g., other exogenous stem cells, pluripotent cells, somatic cells, and/or combinations thereof. In at least one embodiment, one or more therapeutic, prophylactic, and/or diagnostic agents is administered prior to, in conjunction with, and/or subsequent to treatment with one or more one or more d-MAPPS compositions (e.g., d-MAPPS solutions that include MSC, MSC-Exos, and/or one or more MSC-sourced growth factors and/or immunoregulatory proteins).

In other embodiments, one or more therapeutic active agents such as an anti-glaucoma agent, an anti-angiogenesis agent, an anti-infective agent, an anti-inflammatory agent, an analgesic agent, a local anesthetic, a growth factor, an immunosuppressant agent, an anti-allergic agent, an anti-oxidant, and a cytokine are administered prior to, in conjunction with, subsequent to, or alternation with treatment with one or more d-MAPPS compositions (e.g., d-MAPPS solutions that include MSC, MSC-Exos, and/or one or more MSC-sourced growth factors and/or immunoregulatory proteins).

In at least one embodiment, the aforementioned therapeutic, prophylactic and/or diagnostic agents may be administered in a neutral form, or in the form of a pharmaceutically acceptable salt. In at least one example, it may be desirable to prepare a formulation containing a salt of an agent due to one or more of the salt's advantageous physical properties, such as, for example, enhanced stability, a desirable solubility, and/or a desirable dissolution profile.

In at least one embodiment, pharmaceutically acceptable salts are prepared by reaction of the free acid or base forms of an active agent with a stoichiometric amount of the appropriate base or acid in water or in an organic solvent, or in a mixture of the two; generally, non-aqueous media such as, for example, ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts, as well as salts formed by reaction of the drug with a suitable organic ligand (e.g., quaternary ammonium salts). Lists of suitable salts are found, for example, in Adejare et al., Remington: The Science and Practice of Pharmacy (23rd ed.), Academic Press (2020).

In at least one embodiment, one or more d-MAPPS compositions (e.g., d-MAPPS solutions that include MSC, MSC-Exos, and/or one or more MSC-sourced growth factors and/or immunoregulatory proteins) comprise one or more local anesthetics. Non-limiting examples of such local anesthetics include tetracaine, lidocaine, amethocaine, proparacaine, lignocaine, and bupivacaine. In at least one example, one or more additional agents, such as, e.g., a hyaluronidase enzyme, is also added to the one or more d-MAPPS compositions (e.g., d-MAPPS solutions that include MSC, MSC-Exos, and/or one or more MSC-sourced growth factors and/or immunoregulatory proteins) to accelerate and/or improve dispersal of the local anesthetic. In some cases, the active agent is an anti-allergic agent such as olopatadine and/or epinastine.

### Anti-Glaucoma Agents

In some embodiments, the one or more additional active agents is one or more anti-glaucoma agents. Representative anti-glaucoma agents include prostaglandin analogs (such as travoprost, bimatoprost, and latanoprost), beta-andrenergic receptor antagonists (such as timolol, betaxolol, levobetaxolol, and carteolol), alpha-2 adrenergic receptor agonists (such as brimonidine and apraclonidine), carbonic anhydrase inhibitors (such as brinzolamide, acetazolamine, and dorzolamide), miotics (e.g., parasympathomimetics, such as pilocarpine and ecothiopate), seretonergics muscarinics, dopaminergic agonists, and adrenergic agonists (such as apraclonidine and brimonidine).

### Anti-Angiogenesis Agents

In some embodiments, the one or more additional active agents is one or more anti-angiogenesis agents. Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYEOOI) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sirna Therapeutics); multifunctional antiangiogenic agents such as NEOVASTAT® (AE-941) (Aetema Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

### Anti-Infective Agents

In at least one embodiment, one or more d-MAPPS compositions (e.g., d-MAPPS solutions that include MSC, MSC-Exos, and/or one or more MSC-sourced growth factors and/or immunoregulatory proteins) are used in combination with one or more antimicrobial agents. An antimicrobial agent, at least in the context of the present disclosure, is a substance that inhibits the growth of microbes including, for instance, bacteria, fungi, viruses, and/or parasites. Accordingly, antimicrobial agents include, for example, antiviral agents, antibacterial agents, antiparasitic agents, and anti-fungal agents. Non-limiting examples of antiviral agents include, e.g., ganciclovir and acyclovir. Non-limiting examples of antibiotic agents include, for example, aminoglycosides (e.g., streptomycin, amikacin, gentamicin, and tobramycin), ansamycins (e.g., geldanamycin and herbimycin), carbacephems, carbapenems, cephalosporins, glycopeptides (e.g., vancomycin, teicoplanin, and telavancin), lincosamides, lipopeptides (e.g., daptomycin, macrolides such as azithromycin, clarithromycin, dirithromycin, and erythromycin), monobactams, nitrofurans, penicillins, polypeptides (e.g., bacitracin, colistin, and polymyxin B), quinolones, sulfonamides, and tetracyclines.

Other exemplary antimicrobial agents include, for instance, iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

### Anesthetics

In at least one embodiment, one or more d-MAPPS compositions (e.g., d-MAPPS solutions that include MSC, MSC-Exos, and/or one or more MSC-sourced growth factors and/or immunoregulatory proteins) are administered in combination with one or more local anesthetics. A local anesthetic, at least in the context of the present disclosure, is a substance that causes reversible local anesthesia and has the effect of loss of sensation of pain. Non-limiting examples of local anesthetics include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and combinations thereof. In at least another aspect of this embodiment, the one or more d-MAPPS compositions include an anesthetic agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. The concentration of local anesthetics in the one or more d-MAPPS compositions can be therapeutically effective, meaning that the concentration is adequate to provide a therapeutic benefit without inflicting harm to the patient.

Ophthalmic anesthetics are agents that act locally to block pain signals at the nerve endings in the eyes. Some exemplary ophthalmic anesthetics are lidocaine, proparacaine, and tetracaine.

### Anti-Inflammatory Agents

In at least one embodiment, one or more d-MAPPS compositions (e.g., d-MAPPS solutions that include MSC, MSC-Exos, and/or one or more MSC-sourced growth factors and/or immunoregulatory proteins) are administered in combination with one or more anti-inflammatory agents. Anti-inflammatory agents reduce inflammation and include, for instance, steroidal and non-steroidal drugs. Suitable steroidal active agents include, for example, glucocorticoids, progestins, mineralocorticoids, and corticosteroids. Other non-limiting examples of anti-inflammatory agents include triamcinolone acetonide, fluocinolone acetonide, prednisolone, dexamethasone, loteprednol, fluorometholone, ibuprofen, aspirin, and naproxen. Non-limiting examples of immune-modulating drugs include cyclosporine, tacrolimus, and rapamycin. Non-limiting examples of non-steroidal anti-inflammatory drugs (NSAIDs) include ketorolac, nepafenac, and diclofenac.

In at least one embodiment, anti-inflammatory agents are anti-inflammatory cytokines. Non-limiting examples of such cytokines include IL-10, IL-17, TNF-α, TGF-β, IL-35, and others described herein. Anti-inflammatory cytokines in the context of biomaterial implants and tissue grafts are cytokines that induce an anti-inflammatory immune environment or suppress an inflammatory immune environment. Activation of regulatory T cells, Tregs, is involved in the prevention of rejection, and the induction and maintenance of peripheral tolerance of the allograft. Th17 cells are a subset of T helper cells which is characterized by the production of IL-17. Th17 cells have been suggested to play a role in allograft rejection. In some embodiments, cytokines to be added to the one or more d-MAPPS compositions (e.g., d-MAPPS solutions that include MSC, MSC-Exos, and/or one or more MSC-sourced growth factors and/or immunoregulatory proteins) are those that induce Tregs activation (e.g. IL-25) and suppress Th17 activation (e.g., IL-10) for minimizing rejection.

### Growth Factors

In at least one embodiment, one or more d-MAPPS compositions (e.g., d-MAPPS solutions that include MSC, MSC-Exos, and/or one or more MSC-sourced growth factors and/or immunoregulatory proteins) are administered in combination with one or more growth factors. As mentioned above herein, growth factors are proteins and/or glycoproteins capable of stimulating cellular growth, proliferation, and/or cellular differentiation. Non-limiting examples of growth factors include transforming growth factor beta (TGF-β), transforming growth factor alpha (TGF-α), granulocyte-colony stimulating factor (GCSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF8), growth differentiation factor-9 (GDF9), acidic fibroblast growth factor (aFGF or FGF-1), basic fibroblast growth factor (bFGF or FGF-2), epidermal growth factor (EGF), vascular endothelial growth factor (VEGF), and hepatocyte growth factor (HGF).

### Cofactors and Essential Nutrients

In at least one embodiment, one or more d-MAPPS compositions (e.g., d-MAPPS solutions that include MSC, MSC-Exos, and/or one or more MSC-sourced growth factors and/or immunoregulatory proteins) are administered in combination with one or more enzyme cofactors, and/or one or more essential nutrients. Non-limiting examples of such cofactors include vitamin C, biotin, vitamin E, and vitamin K. Non-limiting examples of such essential nutrients include amino acids, fatty acids, etc.

### Cells and Tissues

In at least one embodiment, one or more d-MAPPS compositions (e.g., d-MAPPS solutions that include MSC, MSC-Exos, and/or one or more MSC-sourced growth factors and/or immunoregulatory proteins) comprise at least one eukaryotic cell type, including, for instance, at least one cell type other than one or more types of MSCs. Non-limiting examples of such eukaryotic cell types include non-mesenchymal stem cells, immune cells (e.g., T lymphocytes, B lymphocytes, natural killer cells, macrophages, dendritic cells), and combinations thereof. In at least an additional embodiment, the cells used are cells that dampen one or more inflammation responses (e.g., regulatory T cells). In at least a further embodiment, exosomes are generated ex vivo from one or more types of MSCs.

### Formulations

The d-MAPPS compositions (e.g., d-MAPPS solutions that include MSC, MSC-Exos, and/or one or more MSC-sourced growth factors and/or immunoregulatory proteins) can be administered in concentrated form, diluted with sterile water or buffer, formulated as a gel, ointment, or suspension. It can include additional therapeutic, prophylactic or diagnostic agents, either in the solution, gel, ointment or suspension, or as particles (nanoparticles, liposomes, microparticles) or implants. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

### Solutions, Gels, Ointments, and Suspensions

Numerous ophthalmological formulations are known and available. The d-MAPPS solutions can include sterile filtered amniotic fluid, concentrated or diluted with water, buffered saline, or an equivalent, formed into a gel with a polysaccharide such as alginate or hyaluronic acid, polyvinyl pyrrole, or ointment such as petrolatum or mineral oil, or emulsified with lipid or oil. Ophthalmic emulsions are generally dispersions of oily droplets in an aqueous phase. There should be no evidence of breaking or coalescence.

Ophthalmic suspensions generally contain solid particles dispersed in a liquid vehicle; they must be homogeneous when shaken gently and remain sufficiently dispersed to enable the correct dose to be removed from the container. A sediment may occur, but this should disperse readily when the container is shaken, and the size of the dispersed particles should be controlled. The active ingredient and any other suspended material must be reduced to a particle size small enough to prevent irritation and damage to the cornea.

Ophthalmic ointments are generally sterile, homogeneous, semi-solid preparations intended for application to the conjunctiva or the eyelids. They are usually prepared from non-aqueous bases, e.g., soft paraffin (Vaseline), liquid paraffin, and wool fat. They may contain suitable additives, such as antimicrobial agents, antioxidants, and stabilizing agents.

When the solution is dispensed in a multidose container that is to be used over a period of time longer than 24 hours, a preservative must be added to ensure microbiologic safety over the period of use.

Ideally, the pH of ophthalmic drops should be equivalent to that of tear fluid, which is 7.4. However, the decision to add a buffering agent should be based on stability considerations. The pH selected should be the optimum for both stability of the active pharmaceutical ingredient and physiological tolerance. If a buffer system is used, it must not cause precipitation or deterioration of the active ingredient. The influence on the lachrymal flow should also be taken into account.

Although solutions with the same pH as lacrimal fluid (7.4) are ideal, the outer surfaces of the eye tolerate a larger range, 3.5 to 8.5. The normal useful range to prevent corneal damage is 6.5 to 8.5. The final pH of the solution (e.g., one or more d-MAPPS solutions) is often a compromise, because many ophthalmic drugs have limited solubility and stability at the desired pH of 7.4. Buffers or pH adjusting agents or vehicles can be added to adjust and stabilize the pH at a desired level. Ophthalmic solutions are ordinarily buffered at the pH of maximum stability of the drug(s) they contain. The buffers are included to minimize any change in pH during the storage life of the drug; this can result from absorbed carbon dioxide from the air or from hydroxyl ions from a glass container. Changes in pH can affect the solubility and stability of drugs; consequently, it is important to minimize fluctuations in pH. The buffer system should be designed sufficient to maintain the pH throughout the expected shelf-life of the product, but with a low buffer capacity so that when the ophthalmic solution is instilled into the eye, the buffer system of the tears will rapidly bring the pH of the solution back to that of the tears. Low concentrations of buffer salts are used to prepare buffers of low buffer capacity.

The preparation of aqueous ophthalmic drops (which may be one form of the d-MAPPS solutions disclosed herein) requires careful consideration of the need for isotonicity, a certain buffering capacity, the desired pH, the addition of antimicrobial agents and/or antioxidants, the use of viscosity-increasing agents, and the choice of appropriate packaging. Ophthalmic drops are considered isotonic when the tonicity is equal to that of a 0.9% solution of sodium chloride. The eye can usually tolerate solutions equivalent to 0.5-1.8% of sodium chloride (NaCl).

Solutions that are isotonic with tears are preferred. An amount equivalent to 0.9% NaCl is ideal for comfort and should be used when possible. The eye can tolerate tonicities within the equivalent range of 0.6-2% NaCl without discomfort. There are times when hypertonic ophthalmic solutions are necessary therapeutically, or when the addition of an auxiliary agent required for reasons of stability supersedes the need for isotonicity. A hypotonic ophthalmic solution will require the addition of a substance (tonicity adjusting agent) to attain the proper tonicity range.

The most widely used ophthalmic buffer solutions are boric acid vehicle and Sorensen's modified phosphate buffer. The boric acid vehicle is a 1.9% solution of boric acid in purified water or preferably sterile water. It is isotonic with tears. It has a pH of approximately 5 and is useful when extemporaneously compounding ophthalmic solutions of drugs that are most stable at acid pH. This vehicle does not possess large buffer capacity, but it is sufficient to stabilize pH for the short expiratory periods used for compounded solutions, without overwhelming the natural buffers in lacrimal fluid. The second most commonly used buffer solution is the Sorensen's modified phosphate buffer and is used for drugs needing pH values between the range of 6.5-8.0. This buffer uses two stock solutions, one acidic containing NaH₂PO₄, and one basic containing Na₂HPO₄. The formulas for the stock solutions and their respective proportions used to obtain specific pH values are generally known.

In some instances, the d-MAPPS compositions (e.g., d-MAPPS solutions that include MSC, MSC-Exos, and/or one or more MSC-sourced growth factors and/or immunoregulatory proteins) are distributed or packaged in a liquid form. Alternatively, formulations of the d-MAPPS compositions for ocular administration can be packed as a solid, obtained, for example by lyophilisation of a suitable liquid formulation. The solid can be reconstituted with an appropriate carrier or diluent prior to administration.

The d-MAPPS compositions (for instance, in solution, suspension, and/or emulsion form) for ocular administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

The d-MAPPS compositions (for instance, in solution, suspension, and/or emulsion form) for ocular administration may also contain one or more tonicity agents to adjust the isotonic range of the formulation. Suitable tonicity agents are well known in the art and some examples include glycerin, mannitol, sorbitol, sodium chloride, and other electrolytes. Solutions, suspensions, or emulsions for ocular administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

The d-MAPPS compositions (for instance, in solution, suspension, and/or emulsion form) for ocular administration may also contain one or more excipients known art, such as dispersing agents, wetting agents, and suspending agents.

The ophthalmic drug may be present in its neutral form, or in the form of a pharmaceutically acceptable salt. In some cases, it may be desirable to prepare a formulation containing a salt of an active agent due to one or more of the salt's advantageous physical properties, such as enhanced stability or a desirable solubility or dissolution profile.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

### Particles and Implants Containing One or More Therapeutic, Prophylactic or Diagnostic Agents Dispersed in a Polymer Matrix

Particles can also be formed containing one or more therapeutic, prophylactic or diagnostic agents dispersed or encapsulated in a polymeric matrix. The matrix can be formed of non-biodegradable or biodegradable matrices, although biodegradable matrices are preferred. The polymer is selected based on the time required for in vivo stability, e.g., that time required for distribution to the site where delivery is desired, and the time desired for delivery.

Representative synthetic polymers include: poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly (ethylene glycol), polyalkylene oxides such as poly(ethylene oxide), polyalkylene terephthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), methacrylate), poly(butylmethacrylate), poly(ethyl poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly (butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin and prolamines, for example, zein, and polysaccharides such as alginate, cellulose and polyhydroxyalkanoates, for example, polyhydroxybutyrate.

The in vivo stability of the matrix can be adjusted during the production by using polymers such as polylactide co glycolide copolymerized with polyethylene glycol (PEG). PEG if exposed on the external surface may elongate the time these materials circulate since it is hydrophilic.

Examples of preferred non-biodegradable polymers include ethylene vinyl acetate, poly(meth)acrylic acid, polyamides, copolymers and mixtures thereof.

Particles having an average particle size of between 10 nm and 1000 microns are useful in the compositions described herein. In preferred embodiments, the particles have an average particle size of between 10 nm and 100 microns, more preferably between about 100 nm and about 50 microns, more preferably between about 200 nm and about 50 microns. In certain embodiments, the particles are nanoparticles having a diameter of between 500 and 700 nm. The particles can have any shape but are generally spherical in shape.

Microparticle and nanoparticles can be formed using any suitable method for the formation of polymer micro- or nanoparticles known in the art. The method employed for particle formation will depend on a variety of factors, including the characteristics of the polymers present in the polymer-drug conjugate or polymer matrix, as well as the desired particle size and size distribution. The type of therapeutic, prophylactic or diagnostic agent(s) being incorporated in the particles may also be a factor as some therapeutic, prophylactic or diagnostic agents are unstable in the presence of certain solvents, in certain temperature ranges, and/or in certain pH ranges.

In circumstances where a monodisperse population of particles is desired, the particles may be formed using a method which produces a monodisperse population of nanoparticles. Alternatively, methods producing poly disperse nanoparticle distributions can be used, and the particles can be separated using methods known in the art, such as sieving, following particle formation to provide a population of particles having the desired average particle size and particle size distribution.

Common techniques for preparing microparticles and nanoparticles include, but are not limited to, solvent evaporation, hot melt particle formation, solvent removal, spray drying, phase inversion, coacervation, and low temperature casting. Suitable methods of particle formulation are briefly described below. Pharmaceutically acceptable excipients, including pH modifying agents, disintegrants, preservatives, and antioxidants, can optionally be incorporated into the particles during particle formation.

Implants can be formed from one or more polymers. In preferred embodiments, the implants are intraocular implants. Suitable implants include, but are not limited to, rods, discs, wafers, and the like.

Implants can also be formed from a polymeric matrix having one or more therapeutic, prophylactic or diagnostic agents dispersed or encapsulated therein. The matrix can be formed of any of the nonbiodegradable or biodegradable polymers described above, although biodegradable polymers are preferred. The composition of the polymer matrix is selected based on the time required for in vivo stability, e.g., that time required for distribution to the site where delivery is desired, and the time desired for delivery. Implants can also be formed from blends of polymer-drug conjugates with one or more of the polymers described above herein.

The implants may be of any geometry such as fibers, sheets, films, microspheres, spheres, circular discs, rods, or plaques. Implant size is determined by factors such as toleration for the implant, location of the implant, size limitations in view of the proposed method of implant insertion, ease of handling, etc.

Where sheets or films are employed, the sheets or films will be in the range of at least about 0.5 mm×0.5 mm, usually about 3 to 10 mm×5 to 10 mm with a thickness of about 0.1 to 1.0 mm for ease of handling. Where fibers are employed, the fiber diameter will generally be in the range of about 0.05 to 3 mm and the fiber length will generally be in the range of about 0.5 to 10 mm.

The size and shape of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

Intraocular implants may be spherical or non-spherical in shape. For spherical-shaped implants, the implant may have a largest dimension (e.g., diameter) between about 5 μm and about 2 mm, or between about 10 μm and about 1 mm for administration with a needle, greater than 1 mm, or greater than 2 mm, such as 3 mm or up to 10 mm, for administration by surgical implantation. If the implant is non-spherical, the implant may have the largest dimension or smallest dimension be from about 5 μm and about 2 mm, or between about 10 μm and about 1 mm for administration with a needle, greater than 1 mm, or greater than 2 mm, such as 3 mm or up to 10 mm, for administration by surgical implantation.

The vitreous chamber in humans is able to accommodate relatively large implants of varying geometries, having lengths of, for example, 1 to 10 mm. The implant may be a cylindrical pellet (e.g., rod) with dimensions of about 2 mm×0.75 mm diameter. The implant may be a cylindrical pellet with a length of about 7 mm to about 10 mm, and a diameter of about 0.75 mm to about 1.5 mm. In certain embodiments, the implant is in the form of an extruded filament with a diameter of about 0.5 mm, a length of about 6 mm, and a weight of approximately 1 mg. In some embodiments, the dimension are, or are similar to, implants already approved for intraocular injection via needle: diameter of 460 microns and a length of 6 mm and diameter of 370 microns and length of 3.5 mm.

Intraocular implants may also be designed to be least somewhat flexible so as to facilitate both insertion of the implant in the eye, such as in the vitreous, and subsequent accommodation of the implant. The total weight of the implant is usually about 250 to 5000 μg, more preferably about 500-1000 μg. In certain embodiments, the intraocular implant has a mass of about 500 μg, 750 μg, or 1000 μg.

Implants can be manufactured using any suitable technique known in the art. Examples of suitable techniques for the preparation of implants include solvent evaporation methods, phase separation methods, interfacial methods, molding methods, injection molding methods, extrusion methods, coextrusion methods, carver press method, die cutting methods, heat compression, and combinations thereof. Suitable methods for the manufacture of implants can be selected in view of many factors including the properties of the polymer/polymer segments present in the implant, the properties of the one or more therapeutic, prophylactic or diagnostic agents present in the implant, and the desired shape and size of the implant. Suitable methods for the preparation of implants are described, for example, in U.S. Pat. No. 4,997,652 and U.S. Patent Application Publication No. US 2010/0124565.

In certain cases, extrusion methods may be used to avoid the need for solvents during implant manufacture. When using extrusion methods, the polymer/polymer segments and therapeutic, prophylactic or diagnostic agent are chosen so as to be stable at the temperatures required for manufacturing, usually at least about 85 degrees Celsius. However, depending on the nature of the polymeric components and the one or more therapeutic, prophylactic or diagnostic agents, extrusion methods can employ temperatures of about 25° C. to about 150° C., more preferably about 65° C. to about 130° C.

Implants may be coextruded in order to provide a coating covering all or part of the surface of the implant. Such coatings may be erodible or non-erodible, and may be impermeable, semi-permeable, or permeable to the therapeutic, prophylactic or diagnostic agent, water, or combinations thereof. Such coatings can be used to further control release of the therapeutic, prophylactic or diagnostic agent from the implant.

Compression methods may be used to make the implants. Compression methods frequently yield implants with faster release rates than extrusion methods. Compression methods may employ pressures of about 50-150 pounds per square inch (psi), more preferably about 70-80 psi, even more preferably about 76 psi, and use temperatures of about 0° C. to about 115° C., more preferably about 25° C.

### Methods of Administration

The compositions and methods disclosed herein, such as the d-MAPPS compositions (e.g., d-MAPPS solutions that include MSC, MSC-Exos, and/or one or more MSC-sourced growth factors and/or immunoregulatory proteins), are suitable for any discomfort, pain, dryness, excessive tearing, injuries, infections, burns associated with the eye. In some embodiments, the d-MAPPS compositions (e.g., d-MAPPS solutions that include MSC, MSC-Exos, and/or one or more MSC-sourced growth factors and/or immunoregulatory proteins) are used to alleviate pain, facilitate healing, and/or reduce or inhibit scarring.

The compositions and methods disclosed herein, such as the d-MAPPS compositions (e.g., d-MAPPS solutions that include MSC, MSC-Exos, and/or one or more MSC-sourced growth factors and/or immunoregulatory proteins), are also suitable for prophylactic uses. In some embodiments, the d-MAPPS compositions (e.g., d-MAPPS solutions that include MSC, MSC-Exos, and/or one or more MSC-sourced growth factors and/or immunoregulatory proteins) are used to relieve discomfort associated with extended computer use in human subjects.

Examples of eye disorders that may be treated according to the compositions and methods disclosed herein, such as the d-MAPPS compositions (e.g., d-MAPPS solutions that include MSC, MSC-Exos, and/or one or more MSC-sourced growth factors and/or immunoregulatory proteins), include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, meibomian gland dysfunction, anterior and posterior blepharitis, conjunctival hyperemia, conjunctival necrosis, cicatrical scaring and fibrosis, punctate epithelial keratopathy, filamentary keratitis, corneal erosions, thinning, ulcerations and perforations, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, anterior uveitis, posterior uveitis (including toxoplasmosis), pan-uveitis, an inflammatory disease of the vitreous or retina, endophthalmitis prophylaxis and treatment, macular edema, macular degeneration, age-related macular degeneration, proliferative and nonproliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma and combinations thereof. Other disorders including injury, burn, or abrasion of the cornea, cataracts and age related degeneration of the eye or vision associated therewith.

In some embodiments, the d-MAPPS compositions (e.g., d-MAPPS solutions that include MSC, MSC-Exos, and/or one or more MSC-sourced growth factors and/or immunoregulatory proteins) can be applied to the eye dissolve cataracts, reducing cataracts about 5%, about 10%, about 20%, about 30%, about 40%, about 50%, about 60%, about 70%, about 80%, about 90%, or more than 90%, in size. In other embodiments, the d-MAPPS compositions (e.g., d-MAPPS solutions that include MSC, MSC-Exos, and/or one or more MSC-sourced growth factors and/or immunoregulatory proteins) dissolve cataracts, eliminating the need for an operation to remove cataracts. In some embodiments, the d-MAPPS compositions (e.g., d-MAPPS solutions that include MSC, MSC-Exos, and/or one or more MSC-sourced growth factors and/or immunoregulatory proteins) are used to assist recovery from a cataract removal procedure.

The d-MAPPS compositions (e.g., d-MAPPS solutions that include MSC, MSC-Exos, and/or one or more MSC-sourced growth factors and/or immunoregulatory proteins) may be administered to animals, especially mammalian animals for treating or alleviating pain, disease, disorder, infection, or injury of the eye. Mammalian subjects, include, but are not limited to, humans, primates such as monkeys and apes, canines such as dogs, felines such as cats, bovines such as cows, equines such as horses, swine such as pigs, and rodents such as mice and rats. In some embodiments, the d-MAPPS compositions (e.g., d-MAPPS solutions that include MSC, MSC-Exos, and/or one or more MSC-sourced growth factors and/or immunoregulatory proteins) are used to relieve/treat dry eye, treat eye infection, improve vision, or assist recovery from a surgical procedure on the eye in mammals such as dogs, cats, rabbits, and horses.

Case studies have shown an immediate positive disease modification for patients with mild to moderate and severe dry eye syndrome, glaucoma, Sjogren's syndrome, possible Ankylosing spondylitis and age-related declining vision. Due to the viscosity of at least some of the d-MAPPS compositions (e.g., d-MAPPS solutions that include MSC, MSC-Exos, and/or one or more MSC-sourced growth factors and/or immunoregulatory proteins), drops applied directly onto the eye adhere to the ocular surface longer than common over the counter (“OTC”) artificial tear formulas. The capacity to adhere to the ocular surface is paramount when treating injuries and diseases such as Sjogren's syndrome and chemical burns. Some unexpected results reported in the study were perceptible improvement to clarity of vision which had been diminished in several patients. Relief from varying levels of ocular discomfort or pain was observed. Nine (9) patients were administered Snell Eye Chart exams at the start and completion of the initial 30 day study of the d-MAPPS therapy. Five (5) of the nine demonstrated enriched visual acuity and consistently conveyed improvements in visual clarity, distance and reading ability.

Improvements of one to several lines on the test charts were recorded. Only two patients tested at undetectable improvement levels. Visual acuity appeared to be correlated to the level of corneal integrity of the recipient. This was an unexpected benefit from the d-MAPPS therapy and treatments. Other unexpected benefits were being able to read at night for the first time in years and regaining the visibility required to drive a car. Most participants were able to discontinue or drastically reduce the amount and frequency of using additional applications of artificial tears (“AT”) drops and or alternate curatives. One participant diagnosed with mild dry eye exhibited no signs of the disease at the end of the initial 30 day trial.

### Ocular Burns

In some embodiments, the formulations and methods described herein, such as the d-MAPPS compositions (e.g., d-MAPPS solutions that include MSC, MSC-Exos, and/or one or more MSC-sourced growth factors and/or immunoregulatory proteins), are used for assisting recovery from ocular burns, or from procedures managing ocular burns such as autolimbal or allolimbal transplantation.

Ocular burns such as thermal and chemical burns represent potentially blinding ocular injuries. Thermal burns result from accidents associated with firework explosions, steam, boiling water, or molten metal (commonly aluminum). Chemical burns may be caused by either alkaline or acidic agents. Common alkaline agents include ammonium hydroxide used in fertilizer production, sodium hydroxide (caustic soda) used for cleaning drains and pipes, and calcium hydroxide found in lime plaster and cement.

Alkaline agents are particularly damaging as they have both hydrophilic and lipophilic properties, which allow them to rapidly penetrate cell membranes and enter the anterior chamber. Alkali damage results from interaction of the hydroxyl ions causing saponification of cell membranes and cell death along with disruption of the extracellular matrix. Common acidic agents causing injury include sulphuric acid found in car batteries, sulphurous acid found in some bleaches, and hydrochloric acid used in swimming pools. Acids tend to cause less damage than alkalis as many corneal proteins bind acid and act as a chemical buffer. In addition, coagulated tissue acts as a barrier to further penetration of acid. Acid binds to collagen and causes fibril shrinkage.

Recovery of ocular surface burns depends upon the causative agent and the extent of damage to corneal, limbal, and conjunctival tissues at the time of injury. Damage to intraocular structures influences the final visual outcome. Thus, in some embodiments, the d-MAPPS compositions (e.g., d-MAPPS solutions that include MSC, MSC-Exos, and/or one or more MSC-sourced growth factors and/or immunoregulatory proteins) are used to speed the recovery from an ocular burn.

### Ocular Blast Injuries

Ocular blast injuries can be primary, from the blast wave itself; secondary, from fragments carried by the blast wind; tertiary, due to structural collapse or being thrown against a fixed object; or quaternary, from burns and indirect injuries. In some embodiments, the d-MAPPS compositions (e.g., d-MAPPS solutions that include MSC, MSC-Exos, and/or one or more MSC-sourced growth factors and/or immunoregulatory proteins) are used in the management of injuries inflicted by blasts and explosions for preventative and/or therapeutic purposes.

### Eye Surgery

The d-MAPPS compositions (e.g., d-MAPPS solutions that include MSC, MSC-Exos, and/or one or more MSC-sourced growth factors and/or immunoregulatory proteins) are suitable for use in the management of eye surgeries. Eye surgery, ocular surgery, or ophthalmologic surgery, refers to any surgery that is performed on the eye or its adnexa. Exemplary ocular surgeries include laser eye surgery, cataract removal, glaucoma surgery such as canaloplasty, refractive surgery such as LASIK®, corneal surgery, vitreo-retinal surgery, eye muscle surgery, oculoplastic surgery such as eye lid surgery and orbital surgery, surgery involving the lacrimal apparatus, and eye removal.

In some embodiments, the d-MAPPS compositions (e.g., d-MAPPS solutions that include MSC, MSC-Exos, and/or one or more MSC-sourced growth factors and/or immunoregulatory proteins) are used prior, during or after one or more ocular surgeries. Thus, in some embodiments, the d-MAPPS compositions (e.g., d-MAPPS solutions that include MSC, MSC-Exos, and/or one or more MSC-sourced growth factors and/or immunoregulatory proteins) are used along with one or more systemic drugs. For example, at least some of the d-MAPPS compositions are applied as eye drops whilst the patient is on non-steroidal anti-inflammatory drugs such as ibuprofen.

In some embodiments, the d-MAPPS compositions (e.g., d-MAPPS solutions that include MSC, MSC-Exos, and/or one or more MSC-sourced growth factors and/or immunoregulatory proteins) are used to assist recovery from an ocular surgery. In some embodiments, one or more of such d-MAPPS compositions are used to prevent, reduce, or alleviate one or more symptoms from an ocular surgery. For example, one or more of such d-MAPPS compositions can be used during recovery after a surgical procedure of amniotic membrane graft onto the ocular surface. In some embodiments, one or more of such d-MAPPS compositions are used to prevent one or more potential complications from an ocular surgery such as an infection. In some embodiments, one or more of such d-MAPPS compositions are used to assist local tissue repair, and/or minimize scarring of the surgical site.

### Eye Infections

The formulations are suitable for use in the management of eye infections. Eye infections include infections from bacteria, fungi, and viruses. Eye infections can occur in different parts of the eye and can affect just one eye or both. Exemplary eye infections include conjunctivitis, stye, caratitis, and ocular herpes.

In some embodiments, the d-MAPPS compositions (e.g., d-MAPPS solutions that include MSC, MSC-Exos, and/or one or more MSC-sourced growth factors and/or immunoregulatory proteins) are for prophylactic purposes to prevent an outset of a suspected eye infection. For example, if one person with an eye infection, e.g., conjunctivitis, is identified, anyone who has been recently in contact with that person can use the disclosed formulation for prophylactic purposes. In some embodiments, one or more of such d-MAPPS compositions are used to prevent, reduce, or alleviate one or more symptoms from an eye infection.

### Drug-Induced Eye Conditions

The d-MAPPS compositions (e.g., d-MAPPS solutions that include MSC, MSC-Exos, and/or one or more MSC-sourced growth factors and/or immunoregulatory proteins) are also suitable for use in the management of eye problems that arise as a side effect of using one or more systemic drugs.

Thus, in some embodiments, one or more such d-MAPPS compositions are used prior, during or after taking one or more systemic drugs. Exemplary drugs that can cause ocular side effects include corticosteroids, antihistamines, antipsychotic medications, antimalarials, blood pressure medications, herbal medicines, erectile dysfunction drugs, anticholinergics, immunosuppressants, antibiotics, antiarrhythmic agents, and anti-cancer drugs/treatment. Some specific examples are bisphosphonate, amiodarone, tamsulosin, topiramate, ethambutol, minocycline, cyclosporine and tacrolimus.

Corticosteroids used for many conditions such as asthma, allergies, arthritis and skin conditions can cause swelling in the back of the eye or retina and potentially lead to cataracts. Antihistamines, used for conditions such as allergies, can raise certain patients' risk for glaucoma. Antipsychotic medications, such as THORAZINE® and MELLARIL® can be toxic to the retina. Antimalarials, such as PLAQUENIL® (hydroxychloroquine), used to treat malaria, lupus and rheumatoid arthritis, is a known retinal toxin, and the effects are irreversible. FOSAMAX®, a bisphosphonate that is prescribed for post-menopausal women to prevent calcium bone loss, can cause orbital inflammation, uveitis and scleritis.

Cyclosporine and Tacrolimus, commonly used in patients who have undergone organ or bone marrow transplants, can cause posterior reversible encephalopathy syndrome. These patients will present with bilateral vision loss. Minocycline is a tetracycline derivative and is commonly used to treat acne. Minocycline can cause increased intracranial pressure and papilledema, which can cause permanent vision loss if not reversed. Ethambutol is widely used to treat mycobacterial disease, including tuberculosis. If it is not taken at safe doses, it is an optic nerve toxin. Topiramate (Topamax) is used to treat epilepsy and migraine headaches, and it is used off-label for weight loss. It can cause angle-closure glaucoma soon after starting treatment.

Tamsulosin (Flomax), which is used to treat prostate enlargement and improve urinary flow in men. The well-known syndrome, intraoperative floppy iris syndrome, used to occur only in men who were on medicine to relax their prostate. Women with these drugs can at the time of cataract surgery, make surgical risk much higher. Amiodarone (Cordarone) effectively treats cardiac arrhythmias. It causes the appearance of a whorl in the cornea, which does not usually cause symptoms, although some people can have a little bit of blurred vision.

Anticholinergics, e.g., dicyclomine (BENTYL®), and other drugs with anticholinergic effects, are administered to patients who have stomach conditions that require stomach relaxers and to patients with Parkinson's disease. Young patients taking these drugs will develop difficulty with accommodation. Erectile dysfunction drugs, e.g., sildenafil citrate (VIAGRA®) and tadalafil (CIALIS®) are often prescribed for men with erectile dysfunction. Some of the ocular side effects include blue vision, and ischemic optic neuropathy. Further, blood pressure medications can cause glaucoma.

In some embodiments, the formulations and methods disclosed herein, such as the d-MAPPS compositions (e.g., d-MAPPS solutions that include MSC, MSC-Exos, and/or one or more MSC-sourced growth factors and/or immunoregulatory proteins), are used for treating, alleviating, and/or preventing one or more ocular symptoms that arise as a side effect from taking a systemic drug.

In some embodiments, the formulations and methods disclosed herein, such as the d-MAPPS compositions (e.g., d-MAPPS solutions that include MSC, MSC-Exos, and/or one or more MSC-sourced growth factors and/or immunoregulatory proteins), are used for treating, alleviating, and/or preventing one or more ocular symptoms in patients with ocular graft versus host disease. Ocular Graft Versus Host Disease (GVHD) occurs in patients who have undergone allogenic hematological stem cell transplantation. It can occur in patients who have acute or chronic GVHD, though it is more common in patients with the chronic form. Approximately 40-90% of patients with chronic GVHD will develop ocular symptoms. Exemplary ocular manifestations include moderate to severe keratoconjuncitvitis sicca, bilateral marginal keratitis, anterior uveitis, corneal ulceration or neovascularization. Thus, in some embodiments, the d-MAPPS compositions (e.g., d-MAPPS solutions that include MSC, MSC-Exos, and/or one or more MSC-sourced growth factors and/or immunoregulatory proteins) are suitable for treating, alleviating, and/or preventing keratoconjuncitvitis sicca, bilateral marginal keratitis, anterior uveitis, corneal ulceration or neovascularization. In one embodiment, the d-MAPPS compositions (e.g., d-MAPPS solutions that include MSC, MSC-Exos, and/or one or more MSC-sourced growth factors and/or immunoregulatory proteins) are used to improve vision of patients with ocular graft versus host disease after a bone marrow transplant for leukemia.

### Dosages and Dosing Regimens

Specific d-MAPPS compositions (e.g., d-MAPPS solutions) that include human amniotic fluid, and methods of use thereof, have been developed for topical application to the eye, for the treatment of ocular diseases and injuries including dry eyes, Sjogren's syndrome, cataracts, burns and injuries to the eye tissues. The method involves the management of a specifically formulated diluted sterile decellularized human amniotic fluid applied directly to the eye(s), preferably as a liquid ocular solution, much like a common liquid eye drops, lubricant or gel. The d-MAPPS compositions delivered to the surface of the eye can alleviate or prevent at least one symptom of a number of ocular injuries and diseases, including in addition to chronic dry eye disease, Sjogren's syndrome, and burns or injuries, corneal neovascular disorders, corneal opacities (including corneal haze), prolonged redness and inflammation of the eye(s).

Such d-MAPPS compositions have been tested and shown to contain over 300 human growth factors, which can stimulate the proliferation of stem cells, thereby accelerating healing and contributing to modifying the advancement of disease. Due to the viscosity of at least one of such d-MAPPS compositions, drops applied directly onto the eye adhere to the ocular surface longer than common OTC artificial tear formulas. The capacity to adhere to the ocular surface is paramount when treating injuries and diseases such as Sjogren's syndrome and chemical burns.

Unlike Human Amniotic Membrane (HAM) treatments, in the preferred embodiment, one or more d-MAPPS compositions are provided as a single daily application provided by a licensed ophthalmic profession for in-home use by patients. Therefore, nonsurgical ophthalmologists and optometrists can dispense and oversee the therapy, giving patients greater choices and access to treatment. In addition, unlike the surgical application of HAM, daily applications of such d-MAPPS compositions deliver a sustainable level of beneficial growth factors. Further, the d-MAPPS compositions require much less manipulation during processing and is sterilized without the harsh terminal irradiation or e-beam required for HAM.

As demonstrated by the applications, the concentration and dosage (number of times per day of amount of formulation for period of time) will vary depending on the condition to be treated, the severity of the condition, and the inclusion of other therapeutic, prophylactic or diagnostic agents. The appropriate amounts are determined on an individual basis, measuring response to treatment over time, as demonstrated in the examples. In most cases, two to three drops of solution will be administered once or twice daily as needed.

The dilution ratio of at least some of these d-MAPPS compositions will be dependent on the severity of the disorder or injury; for example, early to moderate dry eye or chronic redness, surface inflammation and, intraocular inflammation may be best treated with a low concentration, whereas, Sjogren's syndrome, severe dry eye, a corneal neovascular disorder, or corneal opacity may dictate a higher concentration of these d-MAPPS compositions.

In the case of sustained or controlled release formulations, ointments, implants or injections into the eye, the dosages will be modified to deliver a therapeutically equivalent amount.

Embodiments of the present disclosure will be further understood by reference to the following non-limiting examples. The examples showing preparation of human amniotic formulation are from U.S. Patent Publication No. US2015/0025366.

### EXAMPLES

### Example 1: Preparation of d-MAPPS Compositions Containing Human Amniotic Fluid

### Materials and Methods

Human amniotic fluid is collected from selected Caesarean sections, which make aspiration of the amniotic fluid in clean condition possible. Then the amniotic fluid is stored in refrigerated condition at 2° C. to 6° C. before the clarification and filtration process. The amniotic fluid is centrifuged at 5,000 to 10,000 rpm for 30 minutes to 1 hour in 50 mL to 250 mL swing out buckets. The supernatant is collected. When collecting the supernatant it is important to avoid detaching or aspirating insoluble components possibly coming from the pellet or from the fatty overlayer. If the supernatant still contains residual insoluble components, it may be pre-filtered with 5 to 10μ cellulose ester capsule pre-filters without TRITON® surfactant to avoid contamination in the filtration process. The liquid phase is collected and filtered with poly ether sulfone 1.0μ capsule filters and the liquid is collected. The liquid is then filtered with poly ether sulfone 0.2μ capsule filter. The filtrate is transferred to vials and sealed with stoppers aseptically. Four samples from the final filtrate are taken to test whether the sterile filtered human amniotic fluid retains growth factors, such as human growth hormone, transforming growth factor beta 1, vascular endothelial growth factor, epidermal growth factor, and transforming growth factor beta 3.

The amniotic fluid from the final filtration is aseptically transferred to syringes or vials, then kept in a deep freezer at about −80° C. to about −20° C. for long term storage. The sterile amniotic fluid is dried in the vial via lyophilisation in a built-in a sterile environment. The lyophilisate derived from the amniotic fluid is reconstituted with sterile water before its injection or topical administration. The lyophilisate can be stored at from +4° C. to about +25° C. (room temperature). All of this operation may be carried out in sterile condition and does not need additional sterilization methods such as a final irradiation.

If needed, the lyophilisate derived from amniotic fluid through lyophilisation may be irradiated by e-beam irradiation or gamma ray irradiation to add another guarantee for the final sterility of the powder. Irradiation of a lyophilisate is much less denaturing for proteins and peptides than irradiating aqueous solutions, because the absence of water considerably reduces the production of reactive superoxide anions and their diffusion during irradiation. Such superoxide anions are the main cause of splitting peptide bonds and chemically modifying amino acids of protein and peptides. After lyophilisation, the amniotic fluid is reconstituted by adding the initial volume of water. After gentle homogenization, the powder is quickly dissolved in about one minute.

### Results

The results show retention of growth factors. The concentration of the growth factors in the sterile filtered amniotic fluid is from about 30 pg/mL to about 2500 pg/mL. Except the vascular endothelial growth factor in sample 2, the concentrations of all the factors in the four samples are in the range of 30-150 pg/mL. Although part of growth differentiation factor 11 is lost in centrifugation and filtration, the final sterile filtered amniotic fluid still retains about 17% to 29% of growth differentiation factor from the raw human amniotic fluid.

The reconstituted amniotic liquid is transparent and may be used for wound healing, cosmetic, orthopedic, or ophthalmic applications, particularly for the treatment of dry eyes.

### Example 2: Treatment of Dry Eye Patients with Amniotic Fluid Solution

### Materials and Methods

d-MAPPS composition was prepared as described in Example 1. d-MAPPS compositions were distributed to select patients suffering from the discomfort and pain often accompanied with dry eyes.

The study was designed for ten patients. Three (3) patients entered too late to effectively chart their results. Ultimately, nine (9) patients were officially enrolled in the study.

Study patients were given a 30 day sample of the d-MAPPS composition and instructed to add the therapy of 1-2 drops of the d-MAPPS composition into both eyes twice daily (a.m. and p.m.), to their current prescribed treatments.

The study included the following visual conditions:
Glaucoma;
Chronic Dry Eye;
Moderate Dry Eye;
Mild Dry Eye;
Sjogren's Disease;
Declining sight; and
Ankylosing spondylitis (possible).

The following observations were tracked and recorded:
OSDI Scores;
Visual Acuity;
Redness;
Staining degree;
Tear Break-up Times;
Appearance;
Artificial Tears frequency of use; and
Patient comments.

The Ocular Surface Disease Index (OSDI) was used to determine the base degree of Dry Eye being experienced by the participants.

### Results

The OSDI scores showed consistent improvement with the addition of the d-MAPPS composition to the daily treatment plans. For example, one patient's base score was 47.7. After 2 weeks of treatment the score was reduced to 35, after 3 weeks; 27. This was the general trend with all participants in the study.

Nine (9) participants demonstrated improvement in their visual acuity and consistently demonstrated improvement in distance, visual clarity and reading ability. Improvements of one to several lines on the Snellen Eye Chart were also recorded. Visual acuity seemed to be correlated to corneal integrity levels.

Accordingly, the d-MAPPS composition tested appeared to have a beneficial impact on improving the corneal epithelial integrity which is important for visual acuity. A common complaint associated with Dry Eye is visual fluctuations. Irregularities in the corneal surface is the most accepted explanation for this phenomena and the d-MAPPS composition demonstrated positive assistance for this particular issue.

Redness of the eye is often associated with severe dry eyes. 8 of the 9 participants in this study were classed with severe dry eye and noted improvement in their level of injection.

Staining levels as rated by the Oxford Method, showed improvement in all participants. One participant listed as moderated dry eye, showed no signs of dry eye after the d-MAPPS composition therapy. Overall, reduction as opposed to elimination in staining would be most accurate in describing the universal results. The d-MAPPS composition demonstrated therapeutic benefit for corneal staining with the unexpected decline in the associated use of artificial tear solution for the participants.

Tear Break Up Times (TBUT) are difficult to measure in the limited time of the study. The return of goblet cells to normal levels required extended management of the patient's disease. However, the d-MAPPS composition that persists on the ocular surface for 90 seconds is likely to have a major contributory effect on the hypermolarity level.

Participants in this study, presented with signs of discomfort, high blinking frequency, squinting and other subnormal appearances to their eyes. Within 2 weeks of initiating therapy with the d-MAPPS composition, 8 of 9 participants had demonstrable improvement in their abnormalities. A surprising benefit of the d-MAPPS composition was the expedience in their desire to reduce the use and frequency of an artificial tears solution. One participate classed as Moderate Dry Eye, quit using her artificial tears 3 weeks into the therapy. Others expressed a desire to reduce or eliminate their use of regular artificial tear solutions as well.

Dry Eye Disease continues to be a condition that has no existing cure but must be managed to provide health, well-being and relief to its victims. There has been a widening gap in the therapeutic treatment options for severe dry eye, particularly for the autoimmune aqueous deficient patient. The d-MAPPS composition demonstrated benefits which could augment or possibly replace current forms of dry eye therapy for these patients, as well as milder forms of the disease.

### Example 3: Comparative Study on the Treatment of Corneal Inflammation

### Materials and Methods

The d-MAPPS composition of Example 1 was dissolved in 1 mL of sterile water to reconstitute 1 mL of the initial sterile filtered amniotic fluid. Two drops were applied on each eye of ten patients suffering from the dry eye syndrome. This treatment was repeated twice per day for ten days. Two other control groups of 10 patients similarly received either their own serum or a serum prepared from cord blood as described by Kyung-Chul Yoon (Umbilical cord and its blood: A perspective on its current and potential use in Ophthalmology, in “Regenerative Medicine Using Pregnancy-Specific Biological Substances,” Springer ed. 2011).

### Results

8 to 10 patients out of 10 in each group declared that they had experienced a significant benefit. For all patients, this clinical improvement was correlated with a partial or complete decrease of their initial corneal inflammation. Nine patient had “severe” dry eye, one was “moderate.” The latter is a 70 year old woman, still working, at a computer all day, who has had dry eye for many years, as well as a long history of allergies, asthma, uses an inhaler as well as an antihistamine, and has had the red eyes associated with this problem. The treatment eliminated the redness and significantly reduced light sensitivity and need for artificial tears. The treatment also restored her ability to read books.

### Example 4: Treatment of Glaucoma

A 90 year-old female patient with glaucoma had been on topical medications for glaucoma. Prior to the study, she had declining vision, persistent central corneal staining and suffered from general dry eye for many years due to incomplete blinking patterns and a tendency to sleep with her eyes partially open. She completed a six-week therapy of d-MAPPS composition drops (twice a day) along with artificial tears. Artificial tears were used eight times a day with a reduced frequency over the period of 6 weeks.

Staining patterns clearly improved after the six-week application but did not resolve completely. Her visual acuity and reading ability improved and as well as her comfort level.

| | | | OD | | OS | |
|---|---|---|---|---|---|---|
| | | | VA; TBUT (sec); | | VA; TBUT (sec); | |
| | OSDI | | Schirmer | | Schirmer | |
| Patient | Before | After | Before | After | Before | After |
| #1 | 52.5 | 21.87 | 20/40; | 20/30; | 20/40; | 20/30 − 2; |
| G. G. | | | 4; — | 2; 12 | 2; — | 2; 12 |

In the above table and in other tables in the Examples, OSDI refers to the dry eye ocular surface disease index; OD refers to oculus dexter, the right eye; OS refers to oculus sinister, the left eye; VA refers to visual acuity; and TBUT refers to the tear break up time, the time it takes for the tear film to start evaporating. The longer it takes for the tear film to break up, the more stable the tear. Schirmer's test determines whether the eye produces enough tears to keep it moist.

### Example 5: Treatment of Age-Related Eye Degeneration

An 81 year-old female patient presented with a poor physical appearance due to closed eyes, minimal eye contact and generally downward posture. Prior to the study, she constantly complained about eye discomfort and sensitivity to light. She had uncontrolled dry eye for the past 10 years and had tried multiple types of therapy with no obvious improvement.

Debris and scurf were observed on her eyelids and eyelashes. She had dementia and was under assisted living condition. She completed a four-week therapy of amniotic fluid drops (twice a day) along with artificial tears. Artificial tears were applied many times a day, depending on the aid's availability with a reduced frequency over the period of four weeks.

Additional methods were used along with the eye drops, including hot lid soaks, gentle cleaning and use of artificial tears of a preservative-free variety.

After the therapy, both the patient and her caregivers noticed significant improvement in the comfort level and life style.

| | | | OD | | OS | |
|---|---|---|---|---|---|---|
| | | | VA; TBUT (sec); | | VA; TBUT (sec); | |
| | OSDI | | Schirmer | | Schirmer | |
| Patient | Before | After | Before | After | Before | After |
| #2 | 58.3 | 33 | 20/40; | 20/30; | 20/40; | 20/30; |
| E. G. | | | 4; 6 (unsure) | 4; 3 | 4; 6 (unsure) | 4; 3 |

### Example 6: Treatment of Moderate Dry Eye

A 71 year-old female patient with moderate dry eye resulted from sustained work at a computer for the past 20 years. She had not attained a very comfortable level with the traditional dry eye treatment and had been seeking better therapy. She had a history of allergies. She completed a four-week therapy of amniotic fluid drops (twice a day) along with artificial tears. She used artificial tears more than eight times a day initially with a gradual declining frequency over time.

After the therapy, she observed great improvement in her eye condition. She reached homeostasis and her eyes were comfortable throughout the day. She was almost free of dry eye towards the end of her therapy period although she felt further improvement if the drops were used.

| | | | OD | | OS | |
|---|---|---|---|---|---|---|
| | | | VA; TBUT (sec); | | VA; TBUT (sec); | |
| | OSDI | | Schirmer | | Schirmer | |
| Patient | Before | After | Before | After | Before | After |
| #3 | 37.5 | 10.41 | 20/20; | 20/20; | 20/20; | 20/20; |
| L. J. | | | 5; 10-11 | 8; 16 | 5; 11 | 8; 16 |

### Example 7: Treatment of Sjorgen's Syndrome

A 77 year-old female patient presented with Sjogren's syndrome and dry eye for 20 years. She had an overall good appearance, mild injection and an anterior blepharitis grade-1 mild stye on superior left lid which was resolving. Prior to the study, she had declining vision and uncomfortable dry eyes. She completed a five-week therapy of d-MAPPS composition drops along with artificial tears. The drops were applied two times a day for the first three weeks, then three times a day for the rest of the period. Artificial tears were applied inconsistently throughout.

This patient improved in a number of areas: comfort, appearance, light-sensitivity, ability to read, general seeing ability, clinical staining signs, and had a number of positive comments to say about the outcome. The patient noted improvement very early in the therapy, and cumulative improvement was appreciated by the patient in the above listed ways as the therapy progressed.

| | | | OD | | OS | |
|---|---|---|---|---|---|---|
| | | | VA; TBUT (sec); | | VA; TBUT (sec); | |
| | OSDI | | Schirmer | | Schirmer | |
| Patient | Before | After | Before | After | Before | After |
| #4 | 70.8 | 31.25 | 20/40; | 20/40 + 2; | 20/50; | 20/30; |
| E. L. | | | 2; 2 | 4; 2 | 2; 2 | 3; 1+ |

### Example 8: Treatment of Dry Eyes

A 64 year-old female patient with dry eyes as a result of her hysterectomy at the age of 38 was treated. She had been diabetic for the past 25 years and had been using metformin. She also had rheumatoid arthritis. Prior to the study, she was less than comfortable in appearance and semi-squinting constantly. She also had complaints of scratchy, sore and burning eyes. She completed a four-week therapy of d-MAPPS composition drops (twice a day) along with artificial tears.

The use of artificial tears declined over time. She had a much improved vision, sunlight sensitivity, comfort levels and appearance after therapy.

| | | | OD | | OS | |
|---|---|---|---|---|---|---|
| | | | VA; TBUT (sec); | | VA; TBUT (sec); | |
| | OSDI | | Schirmer | | Schirmer | |
| Patient | Before | After | Before | After | Before | After |
| #5 | 77 | 8.3 | 20/40−; | 20/40; | 20/50; | 20/40−; |
| L. Z. | | | 3; 3 | 4; 2 | 3; 1 | 4; 2 |

### Example 9: Treatment of Dry Eyes and Mouth

A 40 year-old female patient diagnosed with Sjogren's syndrome in 2003 was treated. She noted dry mouth and subsequently dry eye problems. She was overall in good health with no joint pain or swelling, although her appearance was uncomfortable with constant squinting and blinking. She had severe light sensitivity and burning sensation in her eyes. She preferred to keep her eyes closed if possible. She completed a four-week therapy of d-MAPPS composition drops (twice a day) along with artificial tears, which were applied eight times a day for four weeks.

After the therapy, the patient reported improvement in redness and light sensitivity, comfort level and abilities. Clinical examination identified a significant staining present, suggesting analgesic benefits to the eye drop that suppress the clinical evidence of corneal staining.

| | | | OD | | OS | |
|---|---|---|---|---|---|---|
| | | | VA; TBUT (sec); | | VA; TBUT (sec); | |
| | OSDI | | Schirmer | | Schirmer | |
| Patient | Before | After | Before | After | Before | After |
| #6 | 47.7 | 12.5 | 20/50; | 20/40−; | 20/40; | 20/40; |
| B. M. | | | 1; 1 | 2; 1-2 | 1; 1 | 2; 1-2 |

### Example 10: Treatment of Dry Eye and Light Sensitivity

A 59 year-old female patient with questionable health conditions was treated. She had a recent weight loss with unexplained reasons, chronic back pain from previous injury as well as rheumatoid arthritis. Prior to the study, she had dry eye for more than 10 years along with a severe light sensitivity. She also had mild redness in her eyes, swollen superior lid appearance and clumping of eyelashes due to anterior blepharitis. She complained of severe discomfort in her eyes and had no relief from traditional artificial tears. The chief source of her problem was the meibomian gland dysfunction of the “obstructive” type that rendered her inadequate protection of tear evaporation. She completed a four-week therapy of d-MAPPS composition drops (twice a day) combined with artificial tears. Artificial tears was used 10 times a day but was later reduced to three times a day during the therapy period.

An improvement in appearance and comfort levels was observed upon the completion of the therapy.

| | | | OD | | OS | |
|---|---|---|---|---|---|---|
| | | | VA; TBUT (sec); | | VA; TBUT (sec); | |
| | OSDI | | Schirmer | | Schirmer | |
| Patient | Before | After | Before | After | Before | After |
| #7 | 95.8 | 54 | 20/40; | 20/40; | 20/40; | 20/30; |
| B. D. | | | 5; 4 | 8; 10 | immediately; 5 | 8; 10 |

### Example 11: Treatment of Sjorgen's Syndrome

A 74 year old female patient with Sjogren's syndrome and a severe dry eye condition was treated. She had been forced to compromise some areas in her life such as driving, reading, etc.

After a five-week therapy of d-MAPPS composition drops (twice a day) combined with artificial tears (six to eight times a day), she commented that she was able to drive and that her light sensitivity improved after four and a half weeks after therapy and that she started reading again after years of inability to do so after five weeks after therapy.

| | | | OD | | OS | |
|---|---|---|---|---|---|---|
| | | | VA; TBUT (sec); | | VA; TBUT (sec); | |
| | OSDI | | Schirmer | | Schirmer | |
| Patient | Before | After | Before | After | Before | After |
| #8 | 58.3 | 20.8 | 20/30 (4 | 20/25; | 20/20 − 2 | 20/20; |
| J. P. | | | days into | 4-5; | (4 days into | 4-5; |
| | | | therapy); | 1-2 | therapy); 4; | 1-2 |
| | | | 4; 1 | | 1 | |

### Example 12: Treatment of Glaucoma

An 80 year-old female patient with glaucoma for 10 years, experiencing loss of vision and dry eye, was treated. After a five and a half-week therapy of d-MAPPS composition drops (twice a day) combined with artificial tears (six times a day), her reading ability, eye staining, dry eye symptoms and standard examination scores have improved.

She had been unable to read prior to therapy, and was back to reading after therapy. She had significant central and inferior corneal staining in punctate and patches prior to therapy, and the patches were all cleared with only less serious punctate fine staining after therapy. She had superficial cornea edema appearing three weeks after therapy, which vanished with a mild hypertonic solution. For alleviating edema, the topical glaucoma medication could be removed and changed to oral acetazolamide in the future.

| | | | OD | | OS | |
|---|---|---|---|---|---|---|
| | | | VA; TBUT (sec); | | VA; TBUT (sec); | |
| | OSDI | | Schirmer | | Schirmer | |
| Patient | Before | After | Before | After | Before | After |
| #9 | 54 | 22.7 | 20/60; | 20/50+; | 20/60 + | 20/25; |
| E. D. | | | 4; 9 | 6; 10 | 3; 4; 6 | 6; 12 |

### Example 13: Management and Treatment of GVHD and oGVHD

Approximately 23,000 hematopoietic stem cell transplants (HSCT) are performed annually in the U.S. and it is estimated that chronic graft versus host disease (cGVHD) occurs in 40-70% of the adult allogeneic HSCT. The risk of cGVHD is significantly lower in the pediatric population (22-29%) than in adults. The incidence of ophthalmologic manifestations of disease is very high, reported to occur in 60-90% of patients with diagnosed cGVHD. The most common manifestation of ocular GVHD (oGVHD) is keratoconjunctivitis sicca (KCS), or dry eye, which occurs in 40-75% of all patients with ocular signs and typically develops within 6-9 months post-HSCT. Ocular GVHD (oGVHD) is characterized by a T cell-mediated immune response that leads to immune cell infiltration and inflammation of ocular structures, including the lacrimal glands, eyelids, cornea and conjunctiva.

Dr. Aparna Ramasubramanian, MD, the medical director of retinoblastoma at Phoenix Children's Hospital, as well as a pediatric ophthalmologist and an ocular oncologist, initiated a case study utilizing d-MAPPS composition eye drops.

A 21 year old male underwent a bone marrow transplant following chemotherapy for Acute Myelogenous Leukemia (AML). He developed severe graft versus host disease that involved his skin, oral mucosa, eyes, and liver. He had bilateral blepharconjunctitivitis with severe dry eye and corneal epithelial changes. His Schirmer testing was 0 in both eyes. He was treated with restasis, artificial tears and periodic steroid eye drops. For previous non-healing corneal ulcers he was also treated with contact lens impregnated with amniotic membrane (Prokera) in both eyes. Because of persistent symptoms and superficial punctate keratitis, he was treated with the d-MAPPS composition eye drops 4 times a day. Following 4 weeks of treatment, the patient noted symptomatic improvement with reduced need for artificial tears and improved clarity of vision. His punctate keratitis improved in both eyes and he is continuing the eye drops. His Schirmer test remained at 0, but that is unlikely to improve in view of his severe GVHD.

Ocular GVHD involves both cell-mediated and humoral immunity that leads to infiltration and inflammation of the lacrimal gland, conjunctiva and ocular surface. The d-MAPPS composition eye drops is a combination eye drop that includes, for instance, cytokines, chemokines and growth factors. Due to the immune nature of ocular GVHD, there is potential for such eye drops to be effective in managing this condition. Larger prospective trials will be useful for investigating the utility of d-MAPPS composition eye drops in the management and treatment of ocular GVHD.

### Overall Summary of the Studies

The d-MAPPS compositions (e.g., d-MAPPS solutions that include MSC, MSC-Exos, and/or one or more MSC-sourced growth factors and/or immunoregulatory proteins), including those described above herein administered in drop form, provide definite and real improvement for dry eye, GVHD, and oGVHD. Artificial tears have been the mainstay of dry eye therapy and patients would report that these artificial tears are of no help to their condition, while most clinicians feel they offer no therapeutic benefit. The d-MAPPS compositions feature immediate benefits, e.g., within four days of use, and cumulative improvement as therapy progresses. Patients quickly begin to make lifestyle changes by venturing out more, are not as hindered, note improvements in performance and sustainability during tasks such as using a computer or the ability to stay up later in the evening. Patients' attitudes improve and expectations rise as they sense greater comfort and greater freedom in life, and people are pleased now and at a point of homeostasis. Cosmetic enhancements are noted with all patients due to less injection of bulbar and palpebral conjunctiva. Improvements are noted among a difficult subset of people knows as severe dry eye patients.

Severe dry eye patients often present with compromised appearances due extreme discomfort. Indications of this are habitual squinting, gaze in downward position versus straight ahead, listening to conversation with eyes closed instead of eyes open with good eye contact, high blinking frequency, etc.

A noticeable change in the appearance was apparent in patients in these studies by the end of two weeks of therapy. Other people would comment to these patients that their eyes were looking better. Most patients expressed improvements and increased comfort with therapy. Most patients expressed satisfaction and interest in continuing on the therapy. The majority of the 9 patients studied showed improvements in light sensitivity. One patient reported after two weeks of therapy being able to return to driving after years of avoiding it due to eye discomfort from dryness, sunlight, etc.

The dry eye ocular surface disease index (OSDI) showed a general trend of improvement in OSDI scores as therapy continued.

Frequency of artificial tear use among patients showed a general trend that patients will use less artificial tears after initiating this therapy. This was a surprise early in therapy, often volunteered without prompting. Despite the patients “feeling” like they do not need their previous artificial tears as much as prior to amniotic eye drop therapy, there is objective evidence the patient may benefit from the use more than they are aware. The advantages some artificial tears are meant to provide seem to still benefit the patient, even when the patients are experiencing a new level of soothing and comfort from the use of the d-MAPPS compositions. Supplemental therapy with artificial tears for the moderate dry eye patient, who had no objective clinical evidence of dry eye remaining after three weeks of therapy, showed further improvement in comfort when artificial tears were applied. This observation verifies the hypothesis of what may or may not be accomplished in dry eye therapy. For instance, the forces of evaporation still present challenges to the ocular surface, which are aided by this type of therapy control and management.

Improved reading performance was noted in the majority of the patients, while the other patients had early cataracts developed prior to therapy. Improvements in visual acuity (VA) were noted in the majority of the patients with at least one line on the Snellen chart and in others, two or more. Visual acuity improvements seem closely correlated to corneal integrity levels. When central corneal integrity is compromised as evidenced by corneal staining, visual acuity levels are also compromised. As corneal integrity improves with good therapy, visual acuity also improves as indicated. The d-MAPPS compositions used help heal the corneal surface integrity issues, but are not expected to rehydrate these tissues, and traditional methods of dry eye care may still be advantageous to treat this aspect of dry eye disease. All patients demonstrated improvements in palpebral and bulbar injection levels in essentially all patients within the study.

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

What is claimed is:

1. A method of treating, alleviating, and/or preventing an eye disorder, the method comprising:
administering to an eye of a subject an effective amount of a pharmaceutical composition comprising:
one or more types of mesenchymal stem cells (MSC),
one or more types of MSC-derived exosomes, and/or
one or more MSC-sourced compounds,
thereby treating, alleviating, and/or preventing one or more symptoms of the eye disorder associated with the eye.

2. The method of claim 1, wherein the pharmaceutical composition alters one or more responses of one or more immune cells in the subject, and
wherein the one or more immune cells are selected from the group consisting of: dendritic cells, macrophages, T cells, natural killer (NK) cells, and combinations thereof.

3. The method of claim 1, wherein the one or more MSC-sourced compounds are selected from the group consisting of: MSC-sourced IL-10, MSC-sourced TGF-β, MSC-sourced growth related oncogene (GRO), and combinations thereof.

4. The method of claim 3, wherein the one or more MSC-sourced compounds (i) attenuate expression of one or more co-stimulatory molecules, and/or (ii) suppress production of one or more interleukins in macrophages and dendritic cells.

5. The method of claim 1, wherein the one or more MSC-sourced compounds are selected from the group consisting of: MSC-sourced nitric oxide, MSC-sourced TGF-β, and prostaglandin E2 (PGE2), and combinations thereof.

6. The method of claim 5, wherein the one or more MSC-sourced compounds (i) suppress proliferation and expansion of CD4+ Th1 cells, CD+ Th17 cells, and CD8+ cytotoxic T cells, (ii) decrease cytokine production, and/or (iii) decrease cytotoxicity.

7. The method of claim 1, wherein the one or more MSC-sourced compounds comprise MSC-sourced indoleamine 2,3 dioxygenase (IDO), and wherein the MSC-sourced IDO induces expansion of immunosuppressive Tregs.

8. The method of claim 1, wherein the one or more MSC-sourced compounds comprise one or more microRNAs selected from the group consisting of: microRNA-10a-5p, microRNA-10b-5p, microRNA-191-5p, and combinations thereof.

9. The method of claim 8, wherein the one or more microRNAs (i) prevent apoptosis, and/or (ii) facilitate viability of injured epithelial cells and limbal stem cells.

10. The method of claim 1, wherein the one or more MSC-sourced compounds comprise microRNA-125b, and wherein the microRNA-125b regulates antibody secretion in plasma cells in the eye.

11. The method of claim 1, wherein the pharmaceutical composition is administered as a solution, a suspension, an ointment, a spray, drops, and/or a gel.

12. The method of claim 1, further comprising:
administering to the subject one or more additional agents in combination with the pharmaceutical composition, wherein the one or more additional agents are selected from the group consisting of: an adjuvant, an antigen, an excipient, a vaccine, an allergen, an antibiotic, a gene therapy vector, a kinase inhibitor, a co-stimulatory molecule, a Toll-like receptor (TLR) agonist, a TLR antagonist, a therapeutic agent, a prophylactic agent, a diagnostic agent, an antimicrobial agent, an analgesic, a local anesthetic, an anti-inflammatory agent, an anti-oxidant agent, an immunosuppressant agent, an anti-allergenic agent, an enzyme cofactor, an essential nutrient, a growth factor, and combinations thereof.

13. The method of claim 1, wherein the administering to the eye further comprises:
administering, with the pharmaceutical composition, a pharmaceutically acceptable carrier.

14. The method of claim 1, wherein the eye disorder is ocular graft-versus-host disease (oGVHD), and wherein the one or more symptoms comprises dry eye.

15. The method of claim 1, wherein the eye disorder is selected from the group consisting of: graft-versus-host disease (GVHD), acute GVHD, chronic GVHD, dry eye disease, ocular burns, tears or injury to the eye or associated structures, corneal neovascular disorders, corneal injuries, ocular blast injuries, eye infections, eye surgeries, drug-induced eye conditions, prolonged redness and inflammation of the eye, cataracts, age-related degeneration of the eye or vision associated therewith, amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection, autoimmune uveitis, infectious uveitis, anterior uveitis, posterior uveitis, pan-uveitis, inflammatory disease of the vitreous or retina, endophthalmitis, macular edema, macular degeneration, age related macular degeneration, proliferative and nonproliferative diabetic retinopathy, hypertensive retinopathy, autoimmune disease of the retina, primary and metastatic intraocular melanoma or other intraocular metastatic tumors, open angle glaucoma, closed angle glaucoma, pigmentary glaucoma, and combinations thereof.

16. The method of claim 1, wherein the pharmaceutical composition is administered prior to, in conjunction with, subsequent to, or alternating with, one or more therapeutic, prophylactic, and/or diagnostic agents, and
wherein the one or more therapeutic, prophylactic, and/or diagnostic agents is selected from the group consisting of: an anti-glaucoma agent, an anti-angiogenesis agent, an anti-infective agent, an anti-inflammatory agent, an analgesic agent, a local anesthetic, a growth factor, an immunosuppressant agent, an anti-allergic agent, an anti-oxidant, a cytokine, and combinations thereof.

17. A pharmaceutical composition comprising:
one or more types of mesenchymal stem cells (MSC), one or more types of MSC-derived exosomes, and/or one or more microRNAs sourced from the one or more types of MSC-derived exosomes, and
one or more pharmaceutically acceptable excipients.

18. A kit comprising:
a container containing one or more single, sterile unit doses of the pharmaceutical composition of claim 17.

19. The kit of claim 18, wherein the pharmaceutical composition is in a pharmaceutically acceptable carrier for administration to an eye of a subject.

20. A method of treating, alleviating, or preventing an eye disorder, the method comprising:
administering to the eye of a subject an effective amount of a pharmaceutical composition comprising one or more types of microRNAs, thereby treating, alleviating, and/or preventing one or more symptoms of the eye disorder associated with the eye,
wherein the one or more types of microRNAs are sourced from one or more mesenchymal stem cell (MSC)-derived exosomes.
